(19) United States
(12) Patent Application Publication
(10) Pub. No.: US 2016/0242659 A1
(43) Pub. Date: Aug. 25, 2016

(54) PULSE-WAVE MEASURING MODULE, BIOLOGICAL-INFORMATION MEASURING MODULE, AND ELECTRONIC DEVICE

(71) Applicant: Seiko Epson Corporation, Tokyo (JP)

(72) Inventors: Hideto Yamashita, Suwa-shi (JP); Hironori Hasei, Azumino-shi (JP)

(21) Appl. No.: 15/044,700

(22) Filed: Feb. 16, 2016

(30) Foreign Application Priority Data
Feb. 20, 2015 (JP) 2015-031439
Mar. 9, 2015 (JP) 2015-045583

(51) Int. Cl. A61B 5/024; A61B 5/00

(52) U.S. Cl. CPC A61B 5/02427; A61B 5/02438; A61B 5/681; A61B 2562/0238

(57) ABSTRACT

A biological-information measuring device functioning as a pulse-wave measuring module includes a light emitter configured to emit light to a test object and a light receiver configured to receive reflected light from the test object. The light receiver includes a light detector (a light receiving element) configured to detect the reflected light and an optical filter disposed on the light detector (the light receiving element) and configured by a plurality of layers. A layer most distant from the light detector (the light receiving element) among the plurality of layers of the optical filter is formed by a silicon oxide layer made of silicon oxide.

### CROSS-REFERENCES TO RELATED APPLICATIONS

This application claims priority to Japanese Patents Application No. 2015-031439, filed Feb. 20, 2015, and No. 2015-045583, filed Mar. 9, 2015, all of which are herein incorporated by reference.

### BACKGROUND

1. Technical Field

The present invention relates to a pulse-wave measuring module, a biological-information measuring module, and an electronic device including the pulse-wave measuring module.

2. Related Art

There have been known a measuring device worn on a part such as a wrist by a band or the like to measure biological information such as a pulse wave of a test object and an electronic device of a wristwatch type having a measuring function for the biological information. For example, JP-A-2000-254105 (Patent Literature 1) discloses an arm-wearing-type measuring device mounted with a biological-information measuring module such as a pulse-wave measuring module worn on an arm (a wrist) of a test object (a test subject) to measure biological information such as a pulse wave using an optical pulse wave detection sensor.

Such devices (the measuring device and the electronic device) optically measure a blood flow on a skin surface, which is a measurement target object, and convert the blood flow into a signal to thereby obtain biological information such as a pulse wave. A light emitter, a light receiver, and peripheral components of the light emitter and the light receiver are extremely important elements for obtaining accurate information. In particular, it is important to adopt a configuration that prevent a noise component from being included in light emitted from the light emitter until the light is reflected from an organism (e.g., a skin surface of a test subject) and made incident on the light receiver. It is also necessary to prevent the light from the light emitter from being directly made incident on the light receiver.

When the devices (the measuring device and the electronic device) are used for, for example, a sports-related use, in order to prevent the worn device from affecting the performance of a test object (a test subject), portability, a reduction in size, and a reduction in weight are extremely important viewpoints. When the devices are used for, for example, a medical and health use, a consideration for not imposing a burden on a patient or a test subject is necessary. Portability, a reduction in size, and a reduction in weight are extremely important viewpoints. In this way, in the device worn on apart such as a wrist to obtain biological information, it is demanded to strictly (severely) pursue portability, a reduction in size, and a reduction in weight.

However, in Patent Literature 1, there is no detailed description concerning the light emitter, the light receiver, and the peripheral components of the light emitter and the light receiver in the arm-wearing-type measuring device. Patent Literature 1 does not refer to problems related to the configuration for preventing noise from being included or a configuration for efficiently making light in a desired wavelength band to incident.

### SUMMARY

An advantage of some aspects of the invention is to solve at least a part of the problems and the invention can be implemented as the following forms or application examples.

### Application Example 1

A pulse-wave measuring module according to this application example includes: a light emitter configured to emit light to a test object; and a light receiver configured to receive reflected light from the test object. The light receiver includes: a light detector configured to detect the reflected light; and an optical filter disposed on the light detector and configured by a plurality of layers. A layer most distant from the light detector among the plurality of layers of the optical filter is made of silicon oxide.

According to this application example, the layer most distant from the light detector of the optical filter configuring the light receiver of the pulse-wave measuring module is formed by the silicon oxide layer. The refractive index of the silicon oxide is close to the refractive index of the skin of the test object in contact with the silicon oxide layer. Therefore, a loss of incident light made incident on the optical filter decreases and the optical filter can capture more lights. The optical filter makes light in a desired wavelength band among the captured lights incident on the light detector. The light detector outputs the light as a light reception signal. Consequently, noise relatively decreases and the light reception signal with an improved S/N ratio (signal/noise ratio) is obtained. Therefore, it is possible to provide a pulse-wave measuring module that has less noise and efficiently makes the light in the desired wavelength band incident.

### Application Example 2

In the pulse-wave measuring module according to the application example, it is preferable that the optical filter is configured by the layer made of the silicon oxide and a layer made of silicon nitride.

According to this application example, the optical filter of the pulse-wave measuring module is formed by the silicon oxide layer and the silicon nitride layer. It is possible to attenuate, using the silicon oxide layer of a low-refraction material and the silicon nitride layer of a high-refraction material, light in an unnecessary wavelength band, which is noise in pulse wave measurement, by making use of reflection and interference on a boundary surface between the silicon oxide layer and the silicon nitride layer.

### Application Example 3

In the pulse-wave measuring module according to the application example, it is preferable that a layer nearest from the light detector of the optical filter is the layer made of the silicon oxide, and the layer made of the silicon oxide and the layer made of the silicon nitride are alternately stacked.

According to this application example, in the optical filter of the pulse-wave measuring module, the silicon oxide layer (an oxide layer) and the silicon nitride layer (a nitride layer) are alternately stacked. Apart of the incident light made incident on the optical filter changes to transmitted light and another part of the incident light changes to reflected light on the boundary surface between the oxide layer and the nitride layer. Further, a part of the reflected light is reflected again on the boundary surface and combined with the transmitted light. At this point, phases of the reflected light and the transmitted light of light having a wavelength coinciding with an optical path length of the reflected light coincide with each other and the reflected light and the transmitted light intensify each other. Phases of the reflected light and the transmitted light of light having a wavelength not coinciding with the optical path length of the reflected light do not coincide with each other and the reflected light and the transmitted light weaken each other. Consequently, only the light in the desired wavelength band can reach the light detector. The silicon oxide layer has a high contact property with a substrate (e.g., a silicon substrate) usually used as a base of the light receiver. It is possible to suppress a risk such as peeling of the optical filter from the substrate.

### Application Example 4

In the pulse-wave measuring module according to the application example, it is preferable that the thickness of the layer made of the silicon nitride is smaller than the thickness of the layer made of the silicon oxide.

According to this application example, in the optical filter of the pulse-wave measuring module, the silicon nitride layer is configured by a layer thinner than the silicon oxide layer. The silicon nitride layer is a high-refraction material. The high-refraction material has low light transmittance. Therefore, it is possible to improve light transmittance in the desired wavelength band by setting the thickness of the silicon nitride layer smaller than the thickness of the silicon oxide layer.

### Application Example 5

In the pulse-wave measuring module according to the application example, it is preferable that the thickness of the layer made of the silicon nitride is larger than the thickness of the layer made of the silicon oxide.

According to this application example, in the optical filter of the pulse-wave measuring module, the silicon nitride layer is configured by a layer thicker than the silicon oxide layer. The silicon nitride layer has a higher light blocking rate than the silicon oxide layer. Therefore, it is possible to improve a light blocking rate of light (noise) in an unnecessary wavelength band.

### Application Example 6

In the pulse-wave measuring module according to the application example, it is preferable that the thickness of the optical filter is 0.7 μm or more and 1.0 μm or less.

According to this application example, the optical filter of the pulse-wave measuring module is formed at thickness of 0.7 μm or more. Therefore, it is possible to improve an attenuation region characteristic of the optical filter. Specifically, the number of layers of the optical filter formed by the silicon nitride layer and the silicon oxide layer increases. Therefore, it is possible to improve a light blocking rate for attenuating light in an unnecessary wavelength band (an attenuation region). Conversely, when the thickness of the optical filter exceeds 1.0 μm, a loss of light in the desired wavelength band increases.

### Application Example 7

In the pulse-wave measuring module according to the application example, it is preferable that the thickness of the optical filter is 0.1 μm or more and 0.4 μm or less.

According to this application example, the optical filter of the pulse-wave measuring module is formed at thickness of 0.4 μm or less. Therefore, it is possible to improve a pass band characteristic of the optical filter. Specifically, the number of layers of the optical filter formed by the silicon nitride layer and the silicon oxide layer is small. Therefore, it is possible to reduce a loss of light in the desired wavelength band (a pass band). Conversely, when the thickness of the optical filter is 0.1 μm or less, light in an unnecessary wavelength band is not attenuated because the number of layers of the optical filter is too small.

### Application Example 8

In the pulse-wave measuring module according to the application example, it is preferable that the light receiver is sealed by transparent resin.

According to this application example, the light receiver of the pulse-wave measuring module is covered with the transparent resin for sealing. Therefore, it is possible to improve a waterproof property and an antifouling property of the light receiver. Consequently, it is possible to stably perform accurate measurement of biological information.

### Application Example 9

In the pulse-wave measuring module according to the application example, it is preferable that the transparent resin can come into contact with the test object.

According to this application example, the transparent resin covering the light receiver of the pulse-wave measuring module comes into contact with the skin of the test object, whereby incidence of external light is reduced and the distance between the light receiver and the skin of the test object decreases. Consequently, it is possible to improve detection accuracy of biological information.

### Application Example 10

In the pulse-wave measuring module according to the application example, it is preferable that the pulse-wave measuring module further includes a wall section disposed between the light emitter and the light receiver, and the wall section further projects to the test object side than the light emitter and the light receiver.

According to this application example, the pulse-wave measuring module includes, between the light emitter and the light receiver, the wall section further projecting to the test object side than the light emitter and the light receiver. Therefore, light such as direct light directly made incident on the light receiver from the light emitter is blocked. Consequently, it is possible to prevent light emitted from the light emitter from directly reaching (being made incident on) the light receiver.

### Application Example 11

In the pulse-wave measuring module according to the application example, it is preferable that the pulse-wave measuring module further includes a frame section including the wall section and surrounding the light receiver, and an upper end face of the frame section is higher than an upper surface of the light receiver.

According to this application example, the pulse-wave measuring module includes the frame section higher than the upper surface of the light receiver. Therefore, the frame section and the skin of the test object come into contact with each other, whereby incidence of external light can be prevented. Pressing is stabilized by the frame section and a contact state of the light receiver and the skin of the test object is stable during measurement. Therefore, it is possible to stably detect the reflected light. Further, the optical filter including the silicon oxide layer having a low loss of the reflected light as the outermost layer is used. Therefore, sensitivity is improved and it is possible to perform accurate measurement of a pulse wave.

### Application Example 12

An electronic device according to this application example includes the pulse-wave measuring module according to any of the application examples described above.

According to this application example, the electronic device includes the pulse-wave measuring module that has less noise and efficiently makes the light in the desired wavelength band incident. Therefore, it is possible to provide the electronic device with improved detection accuracy of biological information.

### Application Example 13

A biological-information measuring module according to this application example includes: a first light emitter; a light receiver; and a filter section including a plurality of first convex sections disposed on alight receiving surface of the light receiver, arranged side by side along a first direction from the center of the first light emitter to the center of the light receiver in plan view from a direction perpendicular to the light receiving surface, and projecting from the light receiving surface.

According to this application example, with the filter section including the plurality of first convex sections arranged side by side in parallel to the first direction connecting the center of the first light emitter and the center of the light receiver in plan view from the direction perpendicular to the light receiving surface of the light receiver and projecting from the light receiving surface, it is possible to suppress light from a direction different from the first direction made incident on the light receiver. Further, it is possible to efficiently make light traveling from the first light emitter to the light receiver incident on the light receiver. In other words, with the filter section including the plurality of first convex sections projecting from the light receiving surface, it is possible to limit an incident direction (an incident angle) of the incident light on the light receiver.

By providing the filter section including the first convex sections on the light receiver on a light incident side, it is possible to configure a smaller biological-information measuring module without changing a plane size, that is, without increasing the size of the biological-information measuring module.

The center of the light emitter is the center of a light emitting region in plan view from a direction perpendicular to the light receiving surface. The center of the light receiver is the center of a light receiving region in plan view from the direction perpendicular to the light receiving surface.

### Application Example 14

In the biological-information measuring module according to the application example, it is preferable that the first convex sections extend along the first direction.

According to this application example, with the first convex sections extending along the first direction, it is possible to suppress light from a direction different from the first direction made incident on the light receiver. Further, it is possible to efficiently make light traveling from the first light emitter to the light receiver incident on the light receiver.

### Application Example 15

In the biological-information measuring module according to the application example, it is preferable that the height from the light receiving surface of an end portion convex section located at an end portion of the filter section among the plurality of first convex sections is larger than the height from the light receiving surface of the other first convex sections.

According to this application example, with the high end portion convex section (the first convex section) located at the end portion of the filter section, it is possible to effectively suppress external light or the like from a direction different from the first direction made incident on the light receiver. Further, it is possible to efficiently make light traveling from the first light emitter to the light receiver incident on the light receiver.

### Application Example 16

In the biological-information measuring module according to the application example, it is preferable that the biological-information measuring module further includes a first wall section disposed between the light receiver and the first light emitter, and the height of the first wall section is larger than the height of the first convex sections.

According to this application example, a light component (a noise component) unnecessary for measurement of biological information such as a pulse wave can be cut (blocked) by the high first wall section. Therefore, it is possible to perform more accurate measurement of biological information.

### Application Example 17

In the biological-information measuring module according to the application example, it is preferable that the first convex sections are configured by a stacked body of functional layers.

According to this application example, by configuring the first convex sections with the stacked body of the functional layers, it is possible to form the first convex sections in a process same as, for example, a thin film forming process in forming the light receiver. Therefore, it is possible to efficiently form the first convex sections.

### Application Example 18

In the biological-information measuring module according to the application example, it is preferable that the biological-information measuring module further includes a second light emitter, and the filter section includes a plurality of second convex sections disposed on the light receiving surface of the light receiver, arranged side by side along a second direction from the center of the second light emitter to the center of the light receiver in plan view, and projecting from the light receiving surface.

According to this application example, with, in addition to the first convex sections along the first direction, the plurality of second convex sections arranged side by side along the second direction from the center of the second light emitter to the center of the light receiver and projecting from the light receiving surface, the filter section can suppress light from a direction different from the second direction made incident on the light receiver and can efficiently make light traveling from the second light emitter to the light receiver incident on the light receiver. In this way, even if the directions of the lights emitted from the plurality of light emitters are different, the lights can be efficiently made incident on the light receiver. Therefore, it is possible to make light having higher intensity incident on the light receiver. Further, it is possible to perform incidence limitation for a plurality of incident directions (incident angles) of incident light on the light receiver. Consequently, it is possible to attain further improvement of detection accuracy of the biological-information measuring module.

### Application Example 19

In the biological-information measuring module according to the application example, it is preferable that the second convex sections extend along the second direction.

According to this application example, with the second convex sections extending along the second direction, it is possible to suppress light from a direction different from the second direction made incident on the light receiver. Further, it is possible to efficiently make light traveling from the second light emitter to the light receiver incident on the light receiver.

### Application Example 20

In the biological-information measuring module according to the application example, it is preferable that the biological-information measuring module further includes a second wall section disposed between the light receiver and the second light emitter, and the height of the second wall section is larger than the height of the second convex sections.

According to this application example, a light component (a noise component) unnecessary for measurement of biological information such as a pulse wave can be cut (blocked) by the high second wall section. Therefore, it is possible to perform more accurate measurement of the biological information.

### BRIEF DESCRIPTION OF THE DRAWINGS

The invention will be described with reference to the accompanying drawings, wherein like numbers reference like elements.

FIG. 1A is a schematic exterior view of a biological-information measuring device functioning as an electronic device according to a first embodiment viewed from a front direction side.

FIG. 1B is an exterior view of the biological-information measuring device viewed from an oblique upward direction side in FIG. 1A.

FIG. 2 is an exterior view of the biological-information measuring device viewed from a side direction side in FIG. 1A.

FIG. 3 is a schematic explanatory diagram for explaining wearing of the biological-information measuring device and communication with a portable terminal.

FIG. 4 is a functional block diagram of the biological-information measuring device.

FIG. 5A is a plan view showing a detailed configuration example of a sensor section functioning as a pulse-wave measuring module.

FIG. 5B is a front sectional view showing the detailed configuration example of the sensor section.

FIG. 5C is a partial enlarged view (a front sectional view) of a light receiver.

FIG. 6 is a diagram showing an example of a characteristic of an optical filter.

FIG. 7A is a plan view showing a detailed configuration example of a sensor section functioning as a pulse-wave measuring module according to a second embodiment.

FIG. 7B is a front sectional view showing a detailed configuration example of the sensor section.

FIG. 8A is a plan view showing a configuration example 1 of the sensor section functioning as a biological-information measuring module.

FIG. 8B is a front sectional view showing the configuration example 1 of the sensor section.

FIG. 8C is a partially enlarged view of an A-A section of FIG. 8A.

FIG. 9 is a plan view showing a modification of first convex sections.

FIG. 10A is a plan view showing a configuration example 2 of the sensor section.

FIG. 10B is a plan view showing a configuration example 3 of the sensor section.

FIG. 11A is a plan view showing a configuration example 4 of the sensor section.

FIG. 11B is a plan view showing a configuration example 5 of the sensor section.

FIG. 12 is a plan view showing a configuration example 6 of the sensor section.

FIG. 13A is a sectional view showing a modification 1 of a filter section and equivalent to A-A view of FIG. 8A.

FIG. 13B is a sectional view showing a modification 2 of the filter section and equivalent to the A-A view of FIG. 8A.

FIG. 14 is a sectional view showing a heart-rate monitoring device functioning as a biological-information measuring device of a related art.

FIG. 15 is a perspective view showing a heart-rate monitoring device functioning as a biological-information measuring device according to a fourth embodiment.

FIG. 16 is a side view showing a heart-rate monitoring device functioning as a biological-information measuring device according to a fifth embodiment.

FIG. 17 is a perspective view showing a heart-rate monitoring device functioning as a biological-information measuring device according to a sixth embodiment.

FIG. 18 is a sectional view showing a heart-rate monitoring device functioning as a biological-information measuring device according to a seventh embodiment.

FIG. 19 is a flowchart of a method of manufacturing the biological-information measuring device.

FIG. 20 is a schematic diagram showing a Web page serving as a start point of a health manager in a biological-information measuring device according to an eighth embodiment.

FIG. 21 is a diagram showing an example of a nutrition Web page.

FIG. 22 is a diagram showing an example of an activity level Web page.

FIG. 23 is a diagram showing an example of a mental concentration Web page.

FIG. 24 is a diagram showing an example of a sleep Web page.

FIG. 25 is a diagram showing an example of an everyday activity Web page.

FIG. 26 is a diagram showing an example of a health degree Web page.

FIG. 27 is a partial sectional view showing a modification of a light receiver.

FIG. 28 is a partial sectional view showing a modification of a light emitter.

### DESCRIPTION OF EXEMPLARY EMBODIMENTS

Embodiments of the invention are explained below with reference to the drawings. Note that, in the figures referred to below, layers and members are sometimes shown in scales different from actual scales in order to show the layers and the members in recognizable sizes.

### First Embodiment

### 1. Overall Configuration Example of a Biological-Information Measuring Device Functioning as an Electronic Device

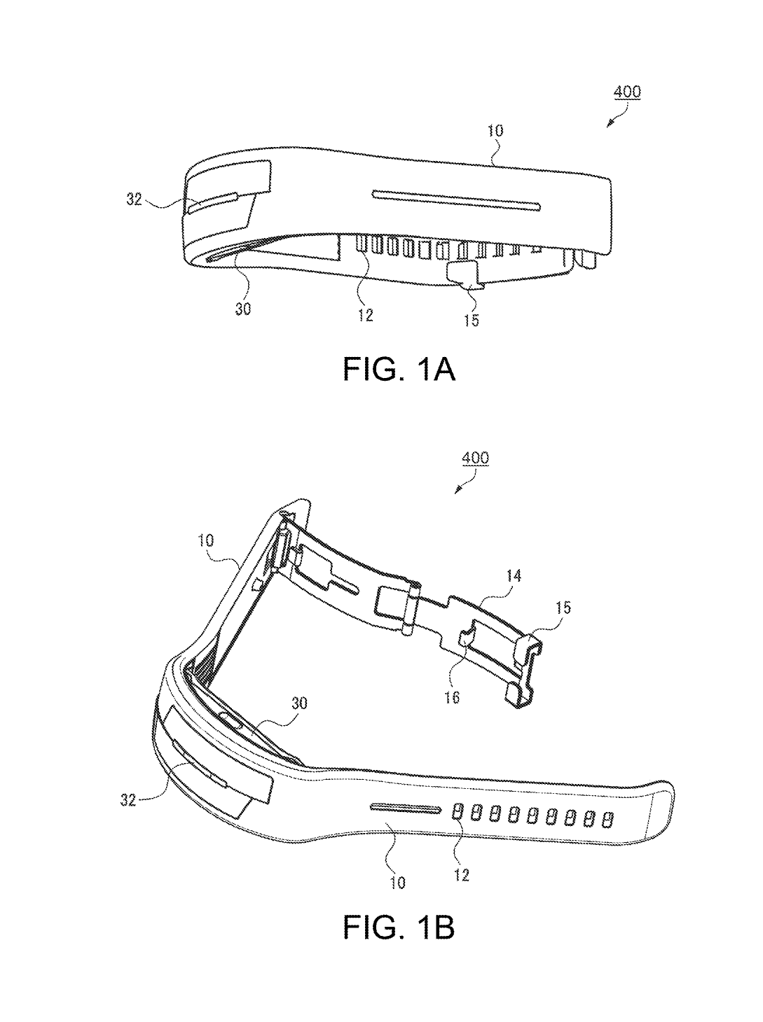

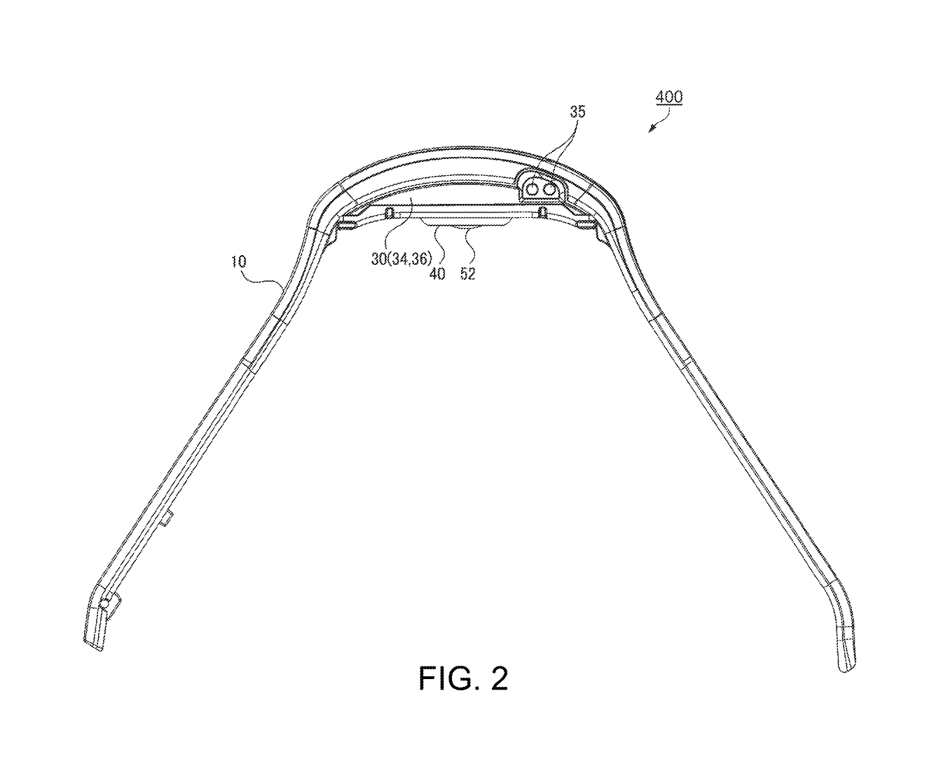

In FIGS. 1A, 1B, and 2, schematic exterior views of a biological-information measuring device according to a first embodiment are shown. FIG. 1A is a diagram of the biological-information measuring device viewed from a front direction side. FIG. 1B is a diagram of the biological-information measuring device viewed from an oblique upward direction side in FIG. 1A. FIG. 2 is a diagram of the biological-information measuring device viewed from a side direction side.

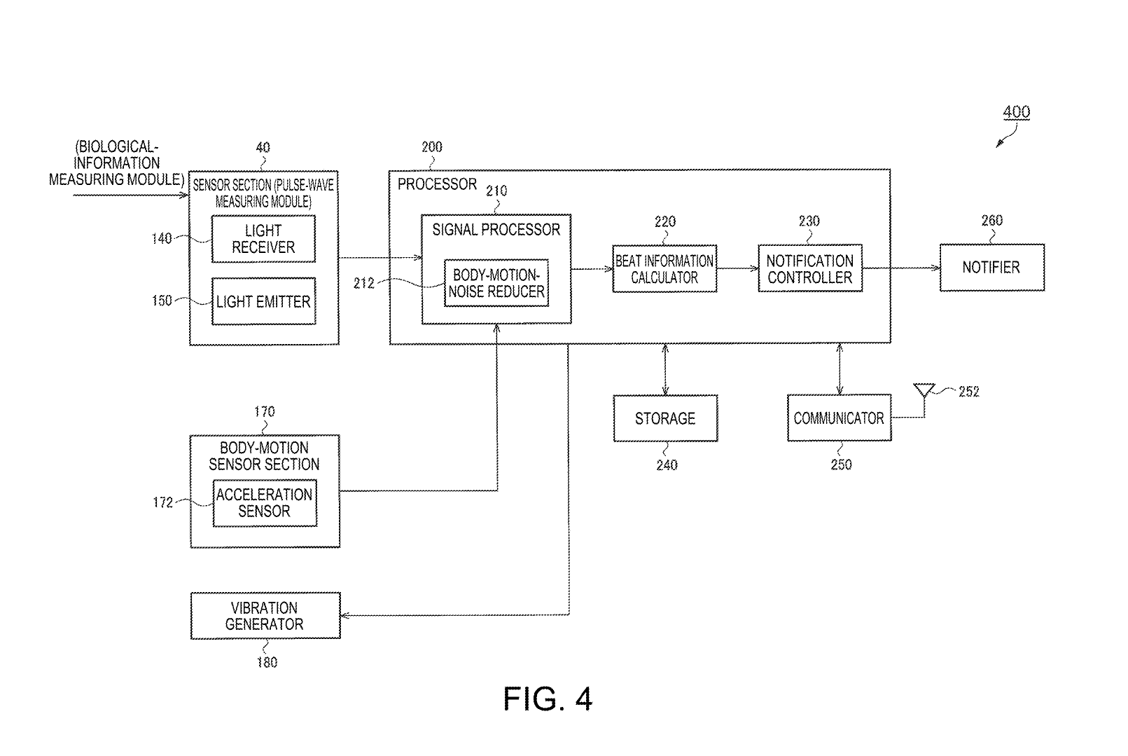

As shown in FIGS. 1A, 1B, and 2, a biological-information measuring device 400 functioning as an electronic device includes a band section 10 and a case section 30. A sensor section 40 functioning as a pulse-wave measuring module is mounted on the biological-information measuring device 400. The case section 30 is attached to the band section 10. The sensor section 40 is provided in the case section 30. The biological-information measuring device 400 includes a processor 200 as shown in FIG. 4 referred to below. The processor 200 is provided in the case section 30. The processor 200 detects, on the basis of a detection signal from the sensor section 40, pulse wave serving as biological information. Note that the biological-information measuring device 400 in this embodiment is not limited to the configuration shown in FIGS. 1A, 1B, and 2. Various modified implementations are possible to, for example, omit a part of components of the biological-information measuring device 400, replace the components with other components, and add other components.

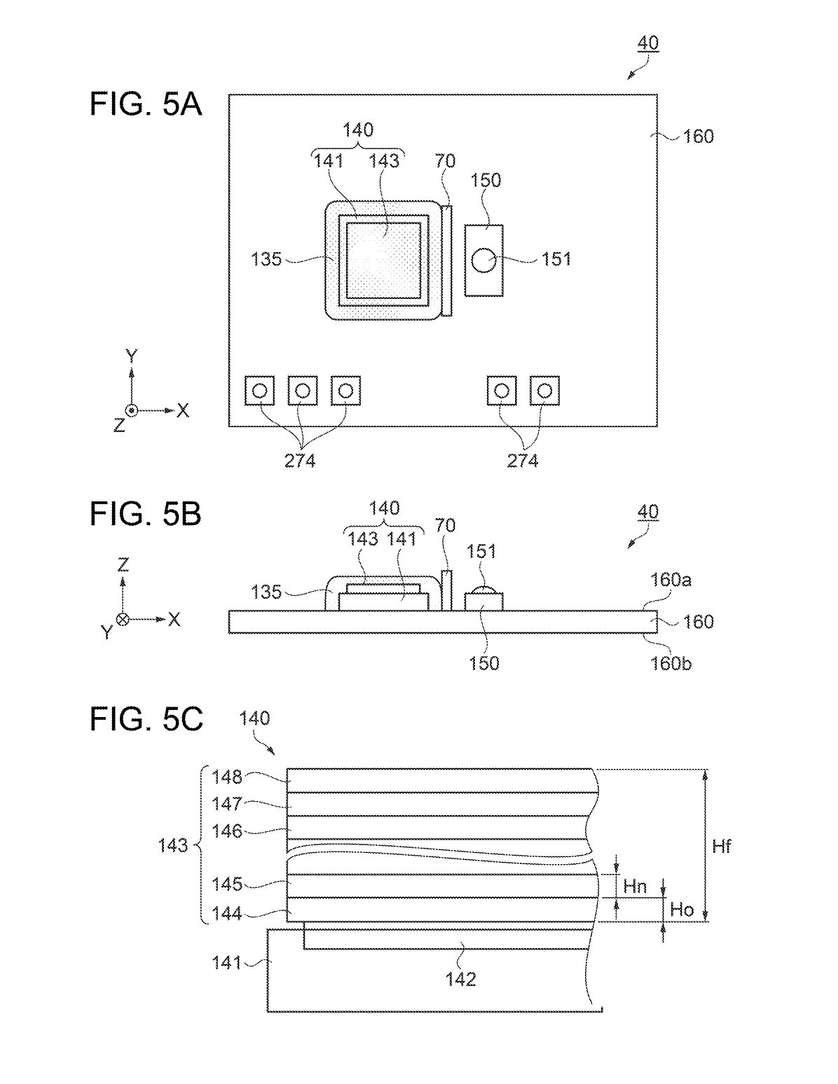

As explained below with reference to FIGS. 5A to 5C, the sensor section 40 functioning as the pulse-wave measuring module is configured from a substrate 160, a light emitter 150, a light receiver 140, a wall section 70 functioning as a frame, and other members. A configuration including these components can be the pulse-wave measuring module (not shown in the figures). Note that the other members are, for example, a projection 52 realized by a light transmitting member. The pulse-wave measuring module according to this embodiment includes the members. That is, a modified implementation is also possible in which the entire sensor section 40 corresponds to the pulse-wave measuring module.

Referring back to FIGS. 1A and 1B and 2, the band section 10 is a section wound around a wrist of a test object (hereinafter referred to as user as well) to wear the biological-information measuring device 400. The band section 10 includes band holes 12 and a buckle section 14. The buckle section 14 includes a band inserting section 15 and a protrusion section 16. The user inserts one end side of the band section 10 into the band inserting section 15 of the buckle section 14 and inserts the protrusion section 16 of the buckle section 14 into the band hole 12 of the band section 10 to wear the biological-information measuring device 400 on the wrist. In this case, the magnitude of pressing (pressing on the wrist surface) of the sensor section 40 explained below is adjusted according to into which of the band holes 12 the protrusion section 16 is inserted.

The case section 30 is equivalent to a main body section of the biological-information measuring section 400. On the inside of the case section 30, various components of the biological-information measuring device 400 such as the sensor section 40 and a processor 200 (see FIG. 4) are provided. That is, the case section 30 is a housing that houses these components. The case section 30 includes, for example, a top case 34 located on the opposite side of the wrist and a bottom case 36 located on the wrist side. Note that the case section 30 does not have to be separated into the top case 34 and the bottom case 36.

A light-emitting window section 32 is provided in the case section 30. The light-emitting window section 32 is formed by a light transmitting member. In the case section 30, a light emitter (an LED; a light emitter for notification different from the light emitter 150 of the pulse-wave measuring module) mounted on a flexible board is provided. Light from the light emitter is emitted to the outside of the case section 30 via the light-emitting window section 32.

As shown in FIG. 2, a terminal section 35 is provided in the case 30. When the biological-information measuring device 400 is mounted on a not-shown cradle, a terminal section of the cradle and the terminal section 35 of the case section 30 are electrically connected. Consequently, a secondary cell (battery) provided in the case section 30 can be charged.

The sensor section 40 functioning as the pulse-wave measuring module detects biological information such as a pulse wave of the test object. For example, the sensor section 40 includes the light receiver 140 and the light emitter 150 as shown in FIGS. 4 and 5 referred to below. The sensor section 40 includes the projection 52 that comes into contact with the skin surface of the test object and applies pressure to the skin surface. In a state in which the projection 52 applies the pressure to the skin surface in this way, the light emitter 150 emits light, the light receiver 140 receives the light reflected by the test object (a blood vessel), and a result of the light reception is output to the processor 200 as a detection signal. The processor 200 detects biological information such as a pulse wave on the basis of the detection signal from the sensor section 40. Note that the biological information serving as a detection target of the biological-information measuring device 400 in this embodiment is not limited to the pulse wave (a pulse rate). The biological-information measuring device 400 may be a device that detects biological information other than the pulse wave (e.g., oxygen saturation in blood, body temperature, and a heartbeat).

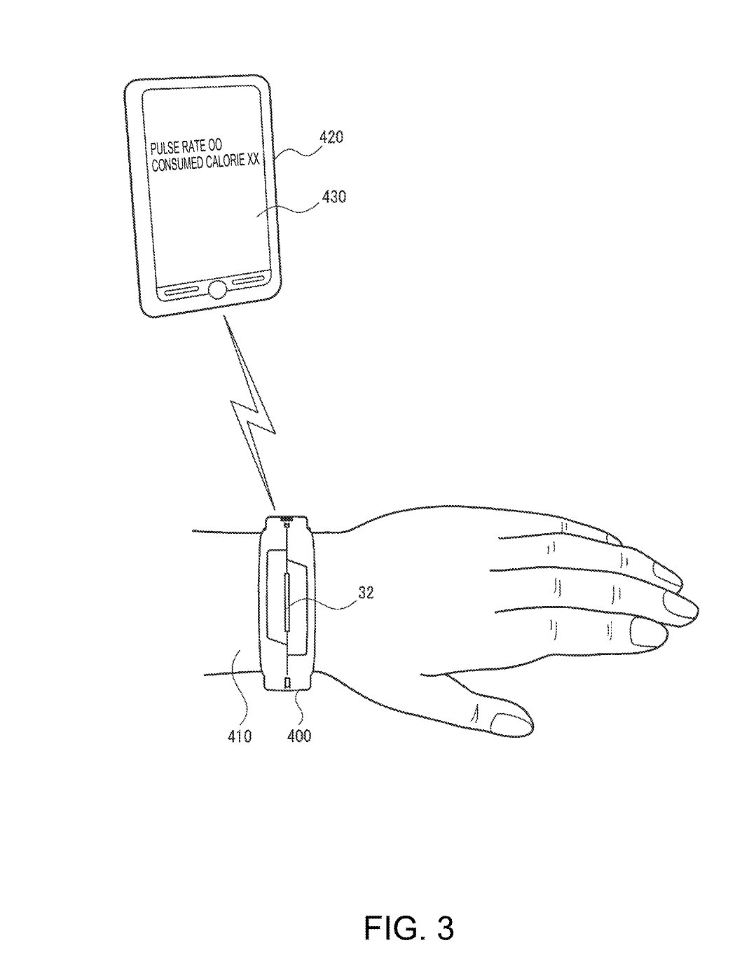

FIG. 3 is a schematic explanatory diagram for explaining wearing of the biological-information measuring device 400 and communication with a terminal device 420. As shown in FIG. 3, the user, who is the test object, wears the biological-information measuring device 400 on a wrist 410 like a watch. As shown in FIG. 2, the sensor section 40 is provided on the surface on the test object side of the case section 30. Therefore, when the biological-information measuring device 400 is worn, the projection 52 of the sensor section 40 comes into contact with the skin surface of the wrist 410 and applies pressure to the skin surface. In that state, the light emitter 150 of the sensor section 40 emits light and the light receiver 140 receives reflected light, whereby biological information such as a pulse wave is detected.

The biological-information measuring device 400 and the terminal device 420 are communicably connected and capable of exchanging data. The terminal device 420 is a portable communication terminal such as a smart phone, a cellular phone, or a future phone. Alternatively, the terminal device 420 may be an information processing terminal such as a tablet computer. As the communicable connection of the biological-information measuring device 400 and the terminal device 420, short-range radio communication such as Bluetooth (registered trademark) can be adopted. Since the biological-information measuring device 400 and the terminal device 420 are communicably connected in this way, various kinds of information such as a pulse rate and a consumed calorie can be displayed on a display section 430 (an LCD, etc.) of the terminal device 420. That is, various kinds of information calculated on the basis of a detection signal of the sensor section 40 can be displayed. Arithmetic processing of the information such as the pulse rate and the consumed calorie may be executed in the biological-information measuring device 400. At least a part of the arithmetic processing may be executed in the terminal device 420.

The light-emitting window section 32 is provided in the biological-information measuring device 400. The biological-information measuring device 400 notifies the user of various kinds of information through light emission (lighting and flashing) of a light emitting body for notification (not shown in the figure). When the user enters a fat burning zone or exits from the fat burning zone in information such as exercise and a consumed calorie, the biological-information measuring device 400 notifies the user of this through light emission of the light emitting body via the light-emitting window section 32. When a mail or the like is received in the terminal device 420, the terminal device 420 notifies the biological-information measuring device 400 of the reception of the mail or the like. Then, the light emitting body of the biological-information measuring device 400 emits light, whereby the reception of the mail or the like is notified to the user.

In this way, in the example shown in FIG. 3, a display section such as an LCD is not provided in the biological-information measuring device 400. Information that needs to be notified by characters, numbers, or the like is displayed on the display section 430 of the terminal device 420. In this way, in the example shown in FIG. 3, a display section such as an LCD is not provided and the biological-information measuring device 400 notifies the user of necessary minimum information through light emission of the light emitting body. Consequently, a reduction in the size of the biological-information measuring device 400 is realized. Since a display section is not provided in the biological-information measuring device 400, it is also possible to improve a fine sight of the biological-information measuring device 400.

A functional block diagram of the biological-information measuring device in this embodiment is shown in FIG. 4. The biological-information measuring device 400 shown in FIG. 4 includes the sensor section 40 functioning as the pulse-wave measuring module (a biological-information measuring module), a body-motion sensor section 170, a vibration generator 180, a processor 200, a storage 240, a communicator 250, an antenna 252, and a notifier 260. Note that the biological-information measuring device 400 in this embodiment is not limited to the configuration shown in FIG. 4. Various modified implementations are possible to, for example, omit a part of components of the biological-information measuring device 400, replace the components with other components, and add other components.

The sensor section 40 functioning as the pulse-wave measuring module detects biological information such as a pulse wave. The sensor section 40 includes the light receiver 140 and the light emitter 150. A pulse wave sensor (a photoelectric sensor) is realized by the light receiver 140, the light emitter 150, and the like. The sensor section 40 outputs, as a pulse wave detection signal, a signal detected by the pulse wave sensor.

The body-motion sensor section 170 outputs a body motion detection signal, which is a signal changing according to a body motion, on the basis of sensor information of various sensors. The body-motion sensor section 170 includes, for example, an acceleration sensor 172 as a body motion sensor. Note that the body-motion sensor section 170 may include a pressure sensor or a gyro sensor as the body motion sensor.

The processor 200 performs various kinds of signal processing and control processing, for example, using the storage 240 as a work region. The processor 200 can be realized by a processor such as a CPU or a logic circuit such as an ASIC. The processor 200 includes a signal processor 210, a beat information calculator 220, and a notification controller 230.

The signal processor 210 performs various kinds of signal processing (filter processing, etc.). The signal processor 210 performs the signal processing on, for example, a pulse wave detection signal from the sensor section 40 and a body motion detection signal from the body-motion sensor section 170. For example, the signal processor 210 includes a body-motion-noise reducer 212. The body-motion-noise reducer 212 performs, on the basis of the body motion detection signal from the body-motion sensor section 170, processing for reducing (removing) body motion noise, which is noise due to a body motion, from the pulse wave detection signal. Specifically, the body-motion-noise reducer 212 performs noise reduction processing using, for example, an adaptive filter.

The beat information calculator 220 performs arithmetic processing of beat information on the basis of, for example, a signal from the signal processor 210. The beat information is information such as a pulse rate. Specifically, the beat information calculator 220 performs frequency analysis processing such as FFT on the pulse wave detection signal after the noise reduction processing in the body-motion-noise reducer 212, calculates a spectrum, and performs processing for calculating a representative frequency in the calculated spectrum as a frequency of a beat. A value obtained by multiplying the calculated frequency with 60 is a pulse rate (a heart rate) to be generally used. Note that the beat information is not limited to the pulse rate itself and may be, for example, other various kinds of information (e.g., a frequency or a cycle of the beat) representing the pulse rate. The beat information may be information representing a state of the beat. For example, a value representing an amount of blood itself may be set as the beat information.

The notification controller 230 controls the notifier 260. The notifier 260 (a notifying device) notifies the user of various kinds of information according to the control by the notification controller 230. As the notifier 260, for example, a light emitting body for notification can be used. In this case, the notification controller 230 controls an electric current flowing to the LED to control lighting, flashing, and the like of the light emitting body. Note that the notifier 260 may be a display section such as an LCD, a buzzer, or the like.

The notification controller 230 performs control of the vibration generator 180. The vibration generator 180 notifies the user of various kinds of information through vibration. The vibration generator 180 can be realized by, for example, a vibration motor (a vibrator). For example, the vibration motor rotates eccentric weights to generate vibration. Specifically, the eccentric weights are attached to both ends of a driving shaft (a rotor shaft) such that the motor itself swings. The vibration of the vibration generator 180 is controlled by the notification controller 230. Note that the vibration generator 180 is not limited to such a vibration motor. Various modified implementations are possible. The vibration generator 180 may be realized by a piezoelectric element or the like.

The vibration by the vibration generator 180 enables, for example, notification of startup at the time of power on, notification of success of initial pulse wave detection, warning at the time when a state in which a pulse wave cannot be detected lasts for a fixed time, notification at the time of movement of a fat burning zone, warning at the time of battery voltage drop, notification of an wakeup alarm, or notification of a mail, a telephone, or the like from a terminal device such as a smart phone. Note that these kinds of information may be notified by the light emitter for notification or may be notified by both of the vibration generator 180 and the light emitter.

The communicator 250 performs the communication processing with the external terminal device 420 as explained with reference to FIG. 3. The communicator 250 performs processing of radio communication conforming to a standard such as Bluetooth. Specifically, the communicator 250 performs reception processing of a signal from the antenna 252 and transmission processing of a signal to the antenna 252. The function of the communicator 250 can be realized by a processor for communication or a logic circuit such as an ASIC.

### 2. Configuration Example of the Sensor Section Functioning as the Pulse-Wave Measuring Module

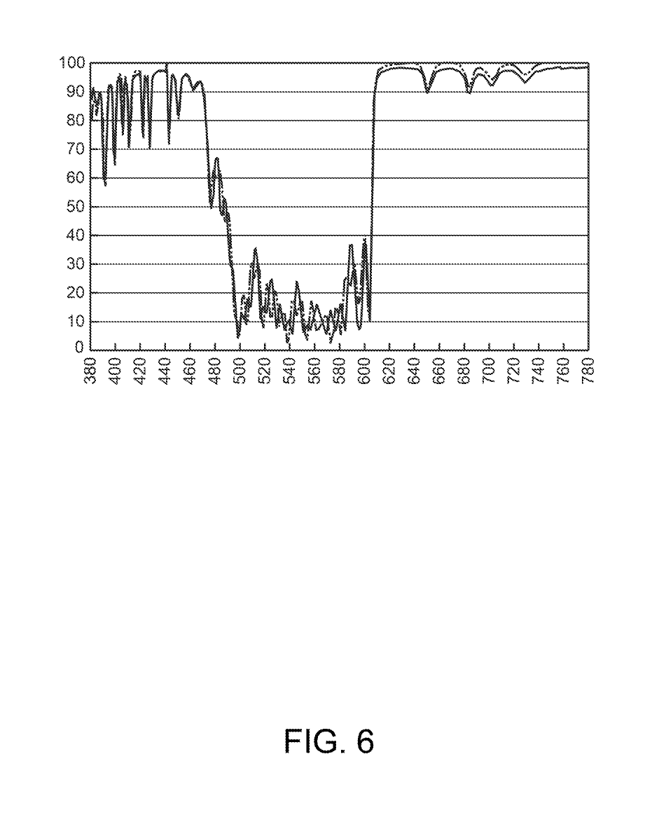

A detailed configuration example of the sensor section 40 functioning as the pulse-wave measuring module is explained with reference to FIGS. 5A to 5C and 6. FIGS. 5A to 5C are diagrams showing a configuration example of the sensor section 40. FIG. 5A is a plan view. FIG. 5B is a front sectional view. FIG. 5C is a partially enlarged view (a front sectional view) of the light receiver 140 configuring the sensor section 40. FIG. 6 is a diagram showing a characteristic of an optical filter 143 provided in the light receiver 140 configuring the sensor section 40.

First, a configuration example 1 of the sensor section 40 is explained with reference to FIGS. 5A to 5C. The sensor section 40 of the configuration example 1 includes the light receiver 140, the light emitter 150, and a wall section 70 disposed between the light receiver 140 and the light emitter 150. The light receiver 140 and the light emitter 150 are arranged side by side at a predetermined interval and are mounted on a supporting surface 160a of the substrate 160 (a sensor substrate) functioning as a supporter. The light emitter 150 emits light to the test object or the like. The light receiver 140 receives reflected light from the test object. For example, when the light emitter 150 emits light and the light is reflected by the test object (e.g., a blood vessel), the light receiver 140 receives and detects the reflected light.

The light emitter 150 can be realized by a light emitting element such as an LED. Note that a dome-type lens 151 (in a broad sense, a condensing lens) functioning as a condensing member provided in the light emitter 150 is a lens for condensing light from an LED chip (in a broad sense, a light-emitting element chip) sealed by resin (sealed by light transmissive resin) in the light emitter 150. That is, in the light emitter 150 of a surface mounting type, the LED chip is disposed below the dome-type lens 151. Light from the LED chip is condensed by the dome-type lens 151 and emitted to the test object. Consequently, the intensity of the light radiated on the test object can be increased. Therefore, it is possible to improve optical efficiency of the pulse-wave measuring module (the sensor section 40) and perform more accurate measurement.

The light receiver 140 includes a light receiving element 142 functioning as a light detector that detects reflected light and the optical filter 143 configured by a plurality of layers disposed on the light receiving element 142. The light receiving element 142 that detects the reflected light can be realized by a photodiode or the like. The optical filter 143 is a wavelength limiting filter (an optical filter layer) that limits a wavelength of light made incident on the light receiving element 142. The light receiver 140 is sealed by transparent resin 135. As a resin material, silicone, epoxy, or the like is used. The resin material is formed by potting or the like. Consequently, a waterproof property and an antifouling property of the light receiver 140 are improved. Further, when the biological-information measuring apparatus 400 is worn on the wrist of the test object (the user), the transparent resin 135 is formed in a shape for enabling a surface on the opposite side of the light receiver 140 to come into contact with the skin of the user. The transparent resin 135 covering the light receiver 140 comes into contact with the skin of the user, whereby incidence of external light is reduced. The distance between the light receiver 140 and the skin of the user decreases. Consequently, it is possible to improve detection accuracy of biological information.

The light receiving element (hereinafter referred to as photodiode as well) 142 is formed on a semiconductor substrate 141 as the photodiode 142. The photodiode 142 is formed as an impurity region by ion injection or the like. For example, the photodiode 142 is realized by PN junction between an N-type impurity region formed on a P substrate and the P substrate. Alternatively, the photodiode 142 is realized by PN junction between a P-type impurity region formed on a deep N well (an N-type impurity region) and the deep N well.

The optical filter 143 is called optical band-pass filter as well. The optical filter 143 is configured by a plurality of layers (a stacked film) on the upper side of the photodiode 142 formed on the semiconductor substrate 141. The stacked film of the optical filter 143 is formed by a layer made of silicon oxide functioning as a low-refraction material and a layer made of silicon nitride functioning as a high-refraction material. Specifically, in the optical filter 143, a layer nearest from the photodiode 142 functioning as a light detector is the layer made of the silicon oxide. The layer made of the silicon oxide and the layer made of the silicon nitride are alternately stacked. Specifically, from a light detector side (the photodiode 142 side), a first layer is a silicon oxide (SiO₂) layer 144 and a second layer is a silicon nitride (Si₃N₄) layer 145. Thereafter, the stacking is alternately repeated in this order. A layer (an uppermost layer) most distant from the photodiode 142 functioning as the light detector among the plurality of layers of the optical filter 143 is formed by a silicon oxide layer 148 made of silicon oxide.

The optical filter 143 can attenuate light (noise) in an unnecessary frequency band made incident on the photodiode 142 making use of reflection and interference on a boundary surface by silicon oxide layers 144, 146, and 148 and silicon nitride layers 145 and 147. For example, when light made incident on the optical filter 143 reaches a boundary surface between the silicon oxide layer 146 and the silicon nitride layer 147, a part of the light changes to transmitted light and another part of the light changes to reflected light. Apart of the reflected light is reflected again on the boundary surface between the silicon nitride layer 147 and the silicon oxide layer 148 and combined with the transmitted light. At this point, phases of the reflected light and the transmitted light of light having a wavelength coinciding with an optical path length of the reflected light coincide with each other and the reflected light and the transmitted light intensify each other. Phases of the reflected light and the transmitted light of light having a wavelength not coinciding with the optical path length of the reflected light do not coincide with each other and the reflected light and the transmitted light weaken each other. Consequently, light in a desired wavelength band can reach the photodiode 142 of the light receiver 140.

The refractive index of the silicon oxide layer formed in the uppermost layer of the optical filter 143 is approximately 1.48. The refractive index of the skin of the test object in contact with the silicon oxide layer 148 is 1.55 and close to the refractive index of the silicon oxide layer. Therefore, a loss (reflection) of incident light made incident on the optical filter 143 decreases and more lights can be captured. The optical filter 143 makes light in a desired wavelength band among the captured lights incident on the light photodiode 142. The photodiode 142 outputs the light as a light reception signal. Consequently, noise relatively decreases and the light reception signal with an improved S/N ratio (signal/noise ratio) is obtained.

As explained above, by configuring the first layer of the optical filter 143 with the silicon oxide layer, adhesion of the light receiver 140 to the semiconductor substrate (e.g., a silicon substrate) 141 usually used as a base is high. It is possible to suppress a risk such as peeling of the optical filter 143 from the semiconductor substrate 141.

A characteristic example (two samples) of the optical filter is explained with reference to a graph shown in FIG. 6. FIG. 6 shows a characteristic of the optical filter configured by five layers as an example of this embodiment. The abscissa indicates a wavelength (nm) of light and the ordinate indicates measurement values of a light blocking rate at respective wavelengths.

As shown in FIG. 6, a desired wavelength band (a transmission band) of the optical filter 143 is approximately 500 nm to 600 nm. Unnecessary wavelength bands (light blocking bands), which are noise in pulse wave measurement, are approximately 300 nm to 500 nm and approximately 600 nm to 1000 nm. In the case of the optical filter configured by the five layers, the light blocking rate of light in the light blocking band is approximately 80% and the light blocking rate (a loss) of light in the transmission band is approximately 15%. In FIG. 6, the characteristic of the optical filter of the five layers is shown as an example in this embodiment. However, by increasing the number of layers of the optical filter, the light blocking rate of the light in the light blocking band increases and the transmittance of the light in the transmission band decreases. By reducing the number of layers of the optical filter, the light blocking rate of the light in the light blocking band decreases and the transmittance of the light in the transmission band increases.

Referring back to FIGS. 5A to 5C, the structure of the optical filter 143 is further explained.

Thickness Hf of the optical filter 143 configured by the plurality of layers is set to 0.7 μm or more and 1.0 μm or less. In the optical filter 143, the silicon oxide layer and the silicon nitride layer are alternately stacked. Therefore, the number of layers of the optical filter can be increased by setting the thickness Hf of the optical filter 143 to 0.7 μm or more. Consequently, it is possible to improve the light blocking rate of the light in the light blocking band. When the thickness Hf of the optical filter 143 exceeds 1.0 μm, the loss of the light in the transmission band increases and the S/N ratio is deteriorated.

The thickness Hf of the optical filter 143 configured by the plurality of layers can be set to 0.1 μm or more and 0.4 μm or less. The number of layers of the optical filter decreases when the thickness Hf of the optical filter 143 is set to 0.4 μm or less. Therefore, it is possible to improve the transmittance of the light in the transmission band. When the thickness Hf of the optical filter 143 is smaller than 0.1 μm, the light blocking rate of the light in the light blocking band decreases and the S/N ratio is deteriorated.

In the optical filter 143 in which the silicon oxide layer and the silicon nitride layer are alternately stacked, thickness Hn of the silicon nitride layer is set smaller than thickness Ho of the silicon oxide layer. The silicon nitride layer functioning as the high-refraction material has low transmittance of light. Therefore, it is possible to improve the transmittance of the light in the transmission band by forming the silicon nitride layer smaller in thickness than the silicon oxide layer.

In the optical filter 143 in which the silicon oxide layer and the silicon nitride layer are alternately stacked, the thickness Hn of the silicon nitride layer can also be set larger than the thickness Ho of the silicon oxide layer. The silicon nitride layer functioning as the high-refraction material has a high light blocking rate of light. Therefore, it is possible to improve the light blocking rate of the light in the light blocking band by forming the silicon nitride layer larger in thickness than the silicon oxide layer.

When a pulse meter is explained as an example of the biological-information measuring device 400, light emitted from the light emitter 150 travels on the inside of the test object, which is a target object, and diffuses or scatters in the outer layer of the skin, the true skin, the subcutaneous tissue, and the like. Thereafter, the light reaches a blood vessel (a region to be detected) and is reflected. At this point, a part of the light is absorbed by the blood vessel. The absorptance of the light in the blood vessel changes because of the influence of a pulse. A light amount of the reflected light also changes. Therefore, the light receiver 140 receives the reflected light and detects a change in the light amount. Consequently, it is possible to detect a pulse rate and the like, which are biological information.

The biological-information measuring device 400 obtains biological information such as a pulse wave and a pulse by optically measuring a blood flow on the skin surface and converting the blood flow into a signal. Therefore, in order to improve accuracy of measurement and portability, it is important to reduce a noise component such as disturbance light in an optical path between the light emitter 150 and the light receiver 140 and reduce light (direct light, etc.) directly made incident on the light receiver 140 from the light emitter 150. From such a viewpoint, the inventors found that it is effective to provide the wall section 70 as a light blocker explained below.

The wall section 70 is mounted on the supporting surface 160a of the substrate 160 between the light receiver 140 and the light emitter 150. The wall section 70 is provided in a wall shape extending in a Y-axis direction along outer peripheral sides on which the light receiver 140 and the light emitter 150 are opposed to each other. The top surface (the upper surface) of the wall section 70 further projects to the test object side than the light emitter 150 and the light receiver 140. The wall section 70 comes into contact with the test object, which is the target object, for example, the skin of the test object on the upper surface and forms a desired space on the upper surfaces of the light receiver 140 and the light emitter 150. The wall section 70 blocks, for example, light such as direct light directly made incident on the light receiver 140 from the light emitter 150 and light such as disturbance light, which is a noise component, made incident from the light receiver 140. In this way, since the wall section 70 is provided, it is possible to prevent the light emitted from the light emitter 150 from directly reaching (being made incident on) the light receiver 140.

Note that, in this configuration example 1, the wall section 70 is explained as the wall-like component extended in the Y-axis direction between the light receiver 140 and the light emitter 150. However, the wall section 70 is not limited to this. For example, as in a sixth embodiment explained below, it is also possible to form the wall section 70 as a frame section surrounding the light receiver and the light emitter like a frame. With such a configuration, effects same as the effects explained above are attained.

A connection terminal 274 electrically connected to a not-shown controller is provided on the supporting surface 160a of the substrate 160 (the sensor substrate) functioning as the supporter. The connection terminal 274 is a terminal for securing electric connection and can be formed by applying gold (Au) plating to a metal layer, for example, a copper (Cu) layer. By providing the connection terminal 274 on the substrate 160, it is possible to compactly connect the supporter and the controller or the like.

As explained above, with the pulse-wave measuring module and the electronic device according to this embodiment, it is possible to obtain effects explained below.

The uppermost layer of the optical filter 143 configured in the light receiver 140 is formed by the silicon oxide layer 148 having a refractive index close to the refractive index of the skin of the test object. Therefore, a loss (reflection) of incident light made incident on the optical filter 143 decreases and more lights can be captured. Consequently, noise relatively decreases and a light reception signal with an improved S/N ratio (signal/noise ratio) is obtained. Therefore, it is possible to provide the pulse-wave measuring module (the sensor section 40) that has less noise and efficiently makes light in a desired wavelength band incident and the electronic device (the biological-information measuring device 400) mounted with the pulse-wave measuring module.

In the optical filter 143, the silicon oxide layer, which is the low-refraction material, and the silicon nitride layer, which is the high-refraction material, are alternately formed in this order from the photodiode 142 side. Therefore, it is possible to cause the light in the desired wavelength band to reach the light detector. The silicon oxide layer 144 has high adhesion to the semiconductor substrate 141. Therefore, it is possible to suppress a risk of peeling of the optical filter 143 from the semiconductor substrate 141.

When the thickness Hf of the optical filter 143 is set to 0.7 μm or more and 1.0 μm or less, it is possible to improve the light blocking rate of light in the light blocking band, which is noise in the pulse wave measurement. Conversely, when the thickness Hf of the optical filter 143 is set to 0.1 μm or more and 0.4 μm or less, it is possible to improve the transmittance of light in the transmission band, which is the desired wavelength band.

When the thickness Hn of the silicon nitride layer is set smaller than the thickness Ho of the silicon oxide layer, it is possible to improve the transmittance of light in the transmission band, which is the desired wavelength band. Conversely, when the thickness Hn of the silicon nitride layer is set larger than the thickness Ho of the silicon oxide layer, it is possible to improve the light blocking rate of light in the light blocking band, which is noise.

### Second Embodiment

A second embodiment of the invention is explained with reference to the drawings.

Note that the same numbers are used for components same as the components in the first embodiment and redundant explanation of the components is omitted. A biological-information measuring device functioning as an electronic device according to this embodiment is different from the biological-information measuring device in the first embodiment in the configuration of a sensor section functioning as a pulse-wave measuring module.

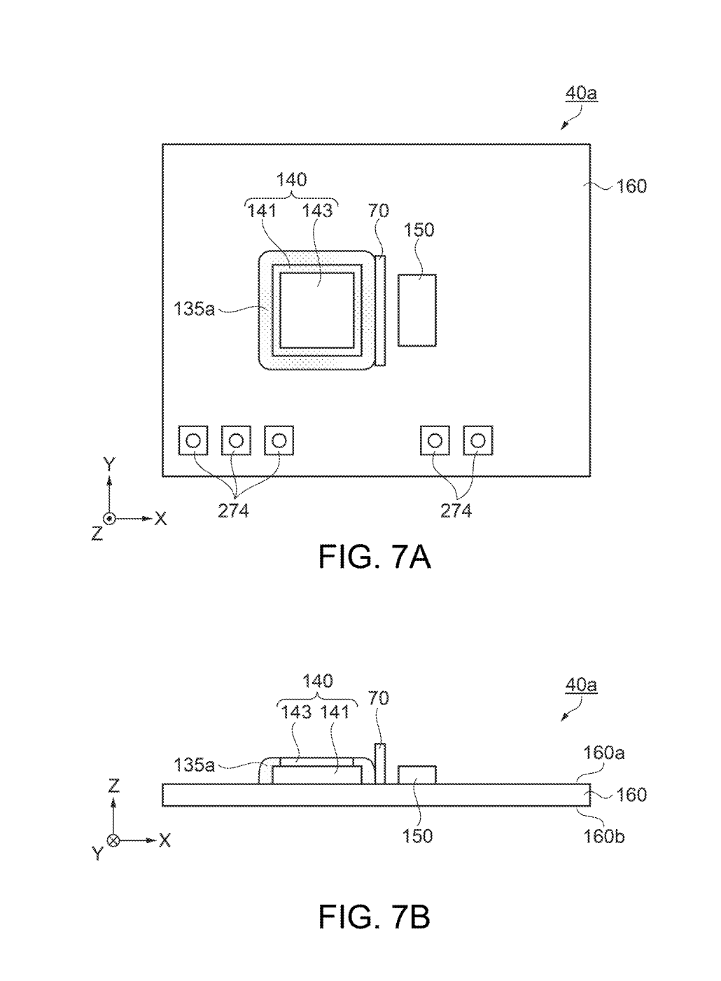

FIGS. 7A and 7B are diagrams showing a configuration example of a sensor section 40a. FIG. 7A is a plan view and FIG. 7B is a front sectional view. A configuration example 2 of the sensor section 40a is explained with reference to the figures. The sensor section 40a of the configuration example 2 includes the light receiver 140, the light emitter 150, and the wall section 70 disposed between the light receiver 140 and the light emitter 150. Note that, in this embodiment, a form is explained in which a condensing lens is not used in the light emitter 150.

The outer periphery of the light receiver 140 is surrounded by transparent resin 135a. Specifically, the transparent resin 135a covers side surfaces of the semiconductor substrate 141 and the optical filter 143. The upper surface of the optical filter 143 (an incident surface of the optical filter) is configured not to be covered with the transparent resin 135a. Consequently, the optical filter 143 and the skin of a test object (a user) adhere to each other not via transparent resin. Therefore, it is possible to suppress an optical loss. Boundary side surfaces between the semiconductor substrate 141 and the optical filter 143 and boundary side surfaces among the layers of the optical filter 143 configured by the plurality of layers are covered with the transparent resin 135a. Therefore, intrusion of moisture and the like from the boundary side surfaces is prevented. The reliability of the pulse-wave measuring module and the electronic device including the pulse-wave measuring module is improved.

### Third Embodiment

### 3. Configuration Example of the Sensor Section Functioning as the Biological-Information Measuring Module

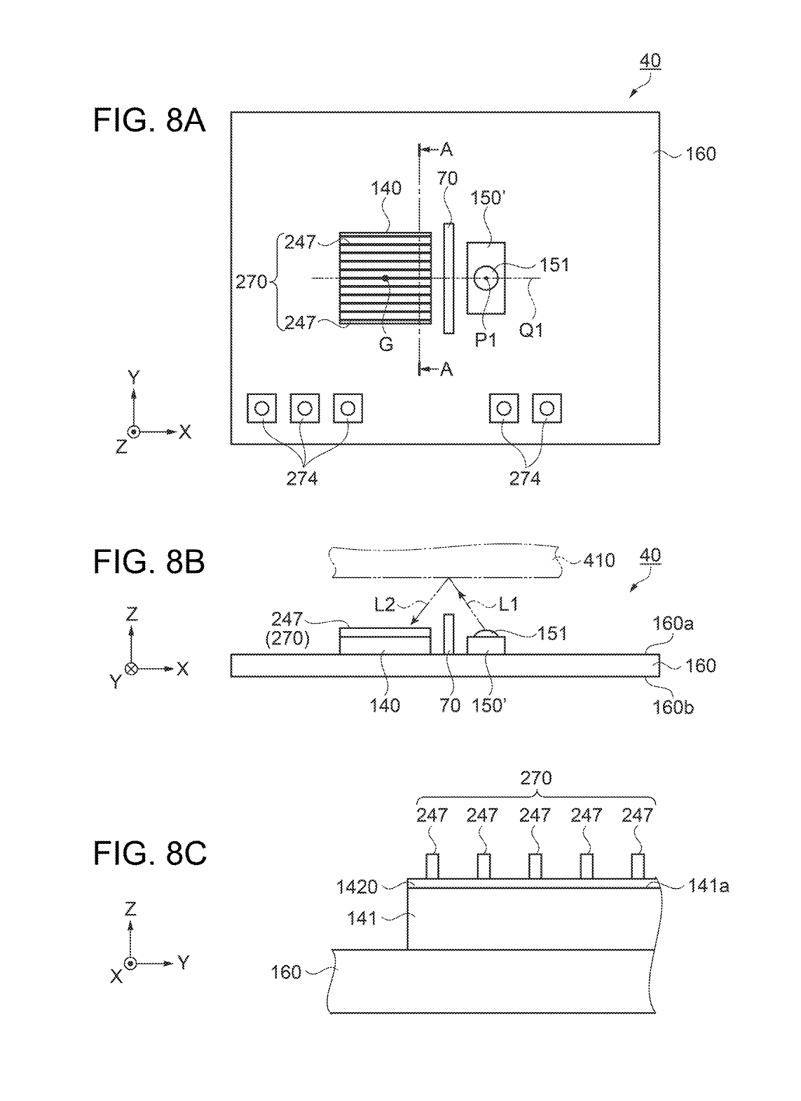

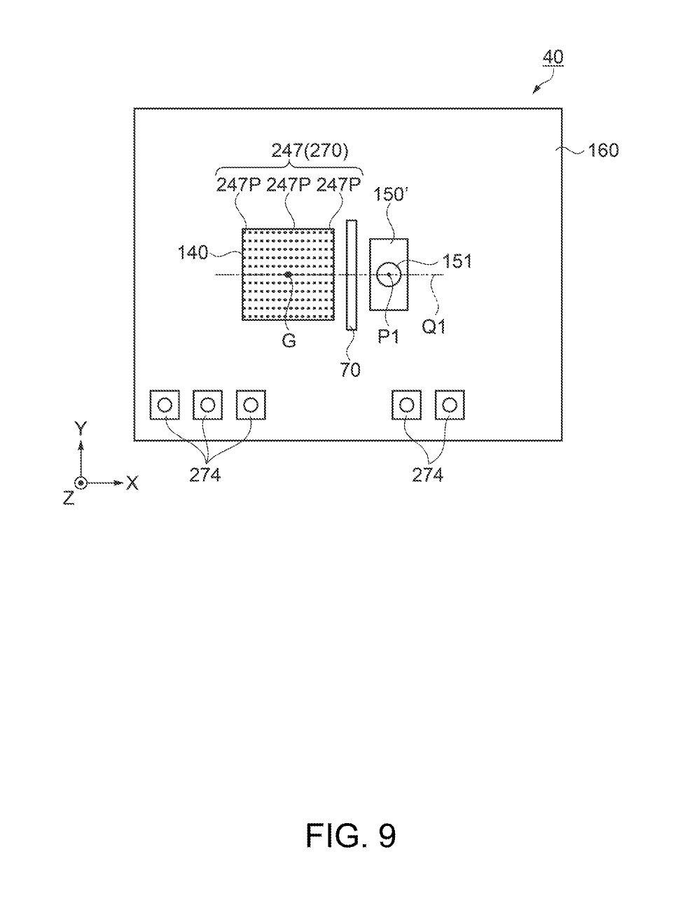

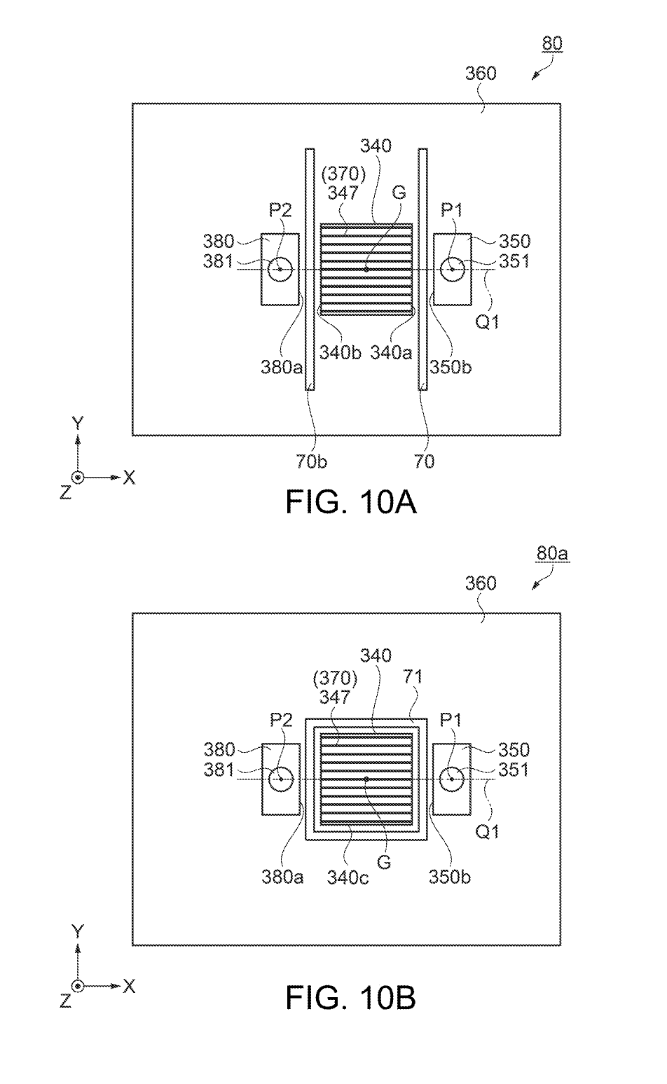

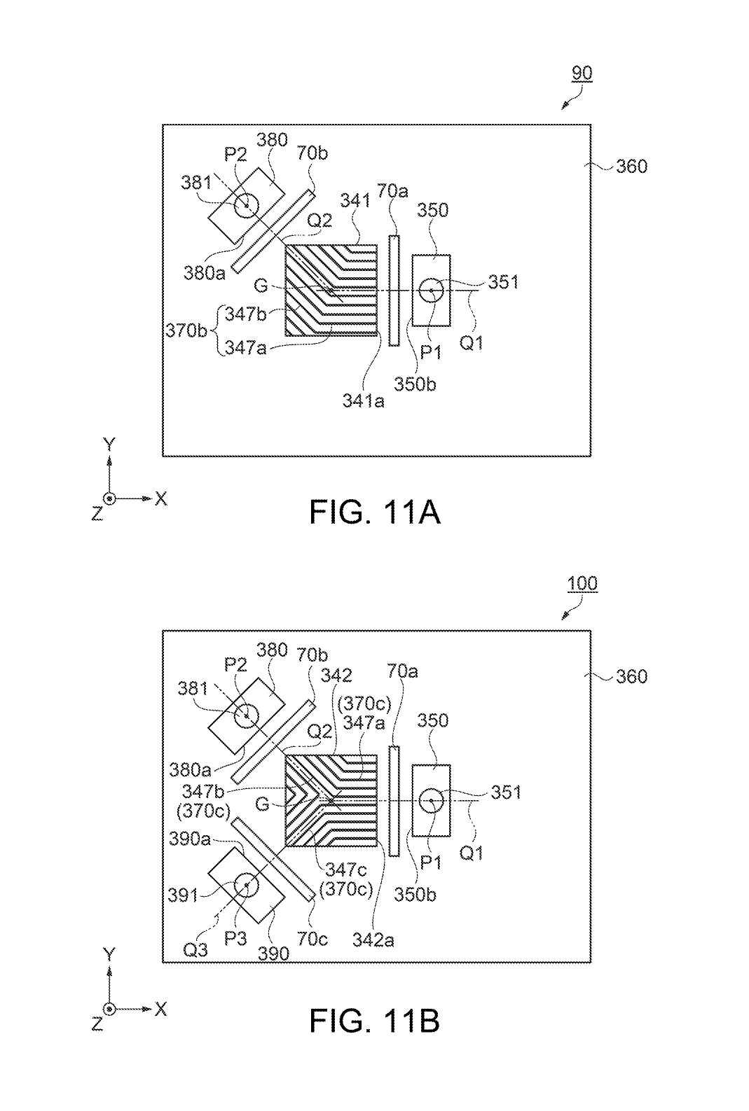

A detailed configuration example of the sensor section 40 functioning as the biological-information measuring module is explained with reference to FIGS. 8A, 8B, 8C, 9, 10A, 10B, 11A, and 11B. FIG. 8A is a plan view showing a configuration example 1 of the sensor section 40. FIG. 8B is a front sectional view. FIG. 8C is a partially enlarged view (a side sectional view) in an A-A section of FIG. 8A. FIG. 9 is a plan view showing a modification of a first convex section. FIG. 10A is a plan view showing a configuration example 2 of the sensor section. FIG. 10B is a plan view showing a configuration example 3 of the sensor section. FIG. 11A is a plan view showing a configuration example 4 of the sensor section. FIG. 11B is a plan view showing a configuration example 5 of the sensor section.

### Configuration Example 1 of the Sensor Section

First, the configuration example 1 of the sensor section 40 is explained with reference to FIGS. 8A to 8C. The sensor section 40 of the configuration example 1 includes the light receiver 140, a first light emitter 150′ functioning as a light emitter, and a first wall section 70 provided between the light receiver 140 and the first light emitter 150′. The light receiver 140 and the first light emitter 150′ are arranged side by side at a predetermined interval and are mounted on the supporting surface 160a of the substrate 160 (the sensor substrate) functioning as the supporter. The first light emitter 150′ and the light receiver 140 are disposed such that a center P1 of the first light emitter 150′ and a center G of the light receiver 140 are located on a first imaginary straight line Q1 along a first direction (an X-axis direction) in plan view of the light receiver 140 viewed from a Z-axis direction. The center of the first light emitter 150′ is the center of a light emitting region of light of the first light emitter 150′ in plan view of the first light emitter 150′ viewed from a direction (the Z-axis direction) perpendicular to an upper surface (a light receiving surface) 141a of a light receiving element configuring the light receiver 140. The center of the light receiver 140 is the center of a light receiving region of the light receiver 140 in plan view of the light receiver 140 viewed from the direction (the Z-axis direction) perpendicular to the upper surface (the light receiving surface) 141a of the light receiving element.

The first light emitter 150′ emits light L1 to a target object (a test object, etc.). The light receiver 140 receives light L2 (reflected light, transmitted light, etc.) reflected on or transmitted through the target object. For example, when the first light emitter 150′ emits the light L1 and the light L1 is reflected by the target object (e.g., a blood vessel), the light receiver 140 receives and detects the light L2 (reflected light of the light L1). The light receiver 140 can be realized by a light receiving element such as a photodiode. The first light emitter 150′ can be realized by a light emitting element such as an LED. For example, the light receiver 140 can be realized by a diode element (not shown in the figures) of PN junction formed on the semiconductor substrate 141. In this case, as a filter section for light, an angle limiting filter for narrowing a light reception angle and a wavelength limiting filter (an optical filter film) for limiting a wavelength of incident light including the light L2 made incident on the light receiving element explained below may be formed on the diode element. In this configuration, an angle limiting filter 270 is provided as the filter section for light.

The angle limiting filter 270 is provided on a protection layer 1420 provided on the upper surface (the light receiving surface) 141a of the light receiving element such as a photodiode. The protection layer 1420 can be formed by an insulating film of SiO₂ or the like. The angle limiting filter 270 includes a plurality of first convex sections 247 arranged side by side along the first imaginary straight line Q1 (the first direction (the X-axis direction)), which connects the center P1 of the first light emitter 150′ and the center G of the light receiver 140, in plan view of the light receiver 140 viewed from the Z-axis direction and projecting from the upper surface (the light receiving surface) 141a of the light receiving element. Each of the first convex sections 247 is formed in a plate wall shape extending along the first direction. The heights of the respective first convex sections 247 from the upper surface 141a of the light receiving element are set substantially the same.

The plurality of first convex sections 247 are formed of a light blocking substance (a light absorbing substance or a light reflecting substance) having a light blocking property for a wavelength detected by the light receiving element such as the photodiode. Although not shown in the figure, the plurality of first convex sections 247 can be configured by alternating stacking, as functional layers, a conductive layer such as an aluminum (light reflecting substance) wiring layer and a conductive plug layer such as a tungsten (light absorbing substance) plug. By configuring the first convex sections 247 with a stacked body of the functional layers, it is possible to form the first convex sections 247 in a process same as a thin-film forming process in forming the light receiver 140 (forming a wiring layer of a circuit of the semiconductor substrate 141) explained below. Therefore, it is possible to efficiently form the first convex sections 247. An aspect ratio of the length of the bottom side (e.g., a longest diagonal line of the bottom surface or the major axis) and the height of the angle limiting filter 270 are set according to a transmission wavelength band of a wavelength limiting filer (not shown in the figures). Opening sections of the angle limiting filter 270 (hollow sections among the first convex sections 247 opposed to one another) may be formed (filled) by an insulating layer of a substance transparent to a wavelength detected by the light receiving element such as the photodiode, for example, SiO₂ (a silicon oxide film).

The angle limiting filter 270 (the first convex sections 247) can be formed by a wiring-layer forming process of another circuit (not shown in the figures) formed on the semiconductor substrate 141. Specifically, the angle limiting filter 270 is formed simultaneously with wiring layer formation of the circuit and formed by the entire or a part of the wiring-layer forming process. For example, the angle limiting filter 270 is formed by aluminum (in a broad sense, light reflecting substance) wiring layer formation by aluminum (Al) sputtering, insulating film formation by SiO₂ deposition, contact formation by tungsten (W) (in a broad sense, light absorbing substance) deposition, or the like. Note that the angle limiting filter 270 is not limited to the aluminum (light reflecting substance) wiring layer and the tungsten (light absorbing substance) contact and may be formed by a wiring layer formed of the light absorbing substance such as tungsten or a contact formed of the light reflecting substance such as aluminum. However, the light blocking property increases when the wring layer or the contact is formed of the light absorbing substance.

Note that the dome-type lens 151 functioning as the condensing member is provided on the first light emitter 150′. The dome-type lens 151 (in a broad sense, a condensing lens) is a lens for condensing light from an LED chip (in a broad sense, a light-emitting element chip) sealed by resin (sealed by light transmitting resin) in the first light emitter 150′. That is, in the first light emitter 150′ of a surface mounting type, the LED chip is disposed below the dome-type lens 151. Light from the LED chip is condensed by the dome-type lens 151 and emitted to the target object. Consequently, the intensity of the light radiated on the target object can be increased. Therefore, it is possible to improve optical efficiency of the sensor section 40 (a light detecting unit functioning as a biological-information measuring module such as a pulse-wave measuring module). It is possible to perform more accurate measurement.

When a pulse meter is explained as an example of the biological-information measuring device, the light L1 emitted from the first light emitter 150′ travels on the inside of the test object, which is the target object, and diffuses or scatters in the outer layer of the skin, the true skin, the subcutaneous tissue, and the like. Thereafter, the light reaches a blood vessel (a region to be detected) and is reflected. At this point, a part of the light is absorbed by the blood vessel. The absorptance of the light in the blood vessel changes because of the influence of a pulse. A light amount of the reflected light also changes. Therefore, the light receiver 140 receives the reflected light (the light L2) and detects a change in the light amount. Consequently, it is possible to detect a pulse rate and the like, which are biological information.

The biological-information measuring device obtains biological information such as a pulse wave and a pulse by optically measuring a blood flow on the skin surface and converting the blood flow into a signal. Therefore, in order to improve accuracy of measurement and portability, it is important to reduce a noise component such as disturbance light in an optical path between the first light emitter 150′ and the light receiver 140 and reduce light (direct light, etc. that are not reflected light) directly made incident on the light receiver 140 from the first light emitter 150′.

According to the above explanation, with the angle limiting filter 270 functioning as the filter section including the plurality of first convex sections 247 extending in parallel in the first direction (the X-axis direction) in which the first light emitter 150′ and the light receiver 140 are arranged side by side and projecting from the upper surface (the light receiving surface) 141a, it is possible to suppress light from a direction different from the first direction made incident on the light receiver 140 and efficiently make the light L2 traveling from the first light emitter 150′ to the light receiver 140 incident on the light receiver 140. In other words, with the plurality of first convex sections 247 projecting from the upper surface (the light receiving surface) 141a of the light receiving element, it is possible to limit an incident direction (an incident angle) of the incident light (the light L2) on the light receiver 140.

By providing the angle limiting filter 270 on the incident surface of the light receiver 140, it is possible to configure the biological-information measuring module without changing a plane size, that is, without increasing the size of the biological-information measuring module.

The first wall section 70 is provided between the light receiver 140 and the first light emitter 150′. The wall section 70 is mounted on the supporting surface 160a of the substrate 160 between the light receiver 140 and the first light emitter 150′. The first wall section 70 has wall surfaces along the respective outer peripheral sides of the light receiver 140 and the first light emitter 150′ disposed to be opposed to each other and is provided in a plate wall shape extending in the Y-axis direction. Note that the height of the first wall section 70 from the supporting surface 160a of the substrate 160 is desirably larger than the height of the first convex sections 247 from the supporting surface 160a of the substrate 160. The first wall section 70 comes into contact with the test object, which is the target object, for example, the skin of the test subject on the top surface (the upper surface) of the first wall section 70 and forms desired spaces on the upper surfaces of the light receiver 140 and the first light emitter 150′. The first wall section 70 can block light such as direct light directly made incident on the light receiver 140 from the first light emitter 150′ and light such as disturbance light, which is a noise component, made incident on the light receiver 140. Since the first wall section 70 is provided in this way, it is possible to prevent light emitted from the first light emitter 150′ from directly reaching (being made incident on) the light receiver 140. Consequently, it is possible to make light with fewer noise components incident on the light receiver 140. It is possible to further improve measurement accuracy of the biological-information measuring module.

The first wall section 70 can be formed by, for example, sheet metal working of a metal plate. If the first wall section 70 is formed by the sheet metal working of the metal plate in this way, it is possible to easily form the first wall section 70 excellent in strength with an inexpensive material. Further, light can be reflected by the first wall section 70 of metal. It is possible to efficiently radiate the light emitted from the first light emitter 150′ on the test object, which is the target object, and efficiently make the reflected light from the test object incident on the light receiver 140. Note that examples of a material of the first wall section 70 other than the metal material include resin such as rubber (including natural resin and synthetic resin). These materials can be inexpensively and easily acquired. It is possible to easily form the first wall section 70 from these materials.

Note that, in this configuration example 1, the first wall section 70 functioning as the frame is explained as the plate wall-like component extended in the Y-axis direction between the light receiver 140 and the first light emitter 150′. However, the first wall section 70 is not limited to this. For example, as in a second embodiment explained below, it is also possible to form the first wall section 70 as a ring-like frame (wall section) surrounding the outer periphery of the light receiver 140 or the first light emitter 150′. With such a configuration, effects same as the effects explained above are attained.

On the supporting surface 160a of the substrate 160 (the sensor substrate) functioning as the supporter, the connection terminal 274 electrically connected to the not-shown controller is provided. The connection terminal 274 is a terminal for securing electric connection and can be formed by applying gold (Au) plating to a metal layer, for example, a copper (Cu) layer. The connection terminal 274 is electrically connected to a connection terminal (not shown in the figures), which is provided on a rear surface 160b, by a through-hole electrode (not shown in the figures) or the like. By providing, on the substrate 160, the connection terminal 274 and the not-shown connection terminal on the rear surface 160b side, it is possible to compactly connect the supporter (the substrate 160) and the not-shown controller or the like.

According to the configurations of the biological-information measuring device and the sensor section 40 functioning as the biological-information measuring module in the first embodiment, with the angle limiting filter 270 including the plurality of first convex sections 247 projecting from the upper surface (the light receiving surface) 141 of the light receiver 140, it is possible to suppress light from a direction different from the first direction made incident on the light receiver 140 and efficiently make the light L2 traveling from the first light emitter 150′ to the light receiver 140 incident on the light receiver 140.

By providing the angle limiting filter 270 on the upper surface 141a of the light receiver 140, it is possible to configure, without changing a plane size, the biological-information measuring module including a desired function such as light blocking of noise light.

### Modification of the First Convex Sections

Note that, the configuration example 1 is explained using the example in which each of the first convex sections 247 is configured in one plate wall shape extending along the first direction. However, the first convex section 247 does not always have to be configured in one plate wall shape. As a modification of the first convex section 247, for example, as shown in FIG. 9, a plurality of convex sections 247p projecting from the upper surface 141a of the light receiving element may be discontinuously and continuously arranged side by side, in other words, the plurality of convex sections 247p may be dotted along the first direction to configure the first convex section 247. The lengths of the respective plurality of convex sections 247p may be the same or may be different.

Note that such a configuration in which the plurality of convex sections 247p are dotted can also be applied in a configuration example 2 and subsequent configuration examples explained below.

### Other Configuration Examples of the Sensor Section

Other configuration examples of the sensor section 40 are explained with reference to FIGS. 10A, 10B, 11A, and 11B. Note that, in FIGS. 10A, 10B, 11A, 11B, 12, 13A, and 13B, the configurations and the disposition of the light receiver, the light emitter, and the wall section are mainly shown. Illustration of the other components is omitted. Components same as the components in the first embodiment are denoted by the same reference numerals and signs. Explanation of the components is sometimes omitted.

### Configuration Example 2

First, a sensor section 80 according to a configuration example 2 is explained with reference to FIG. 10A. In the configuration example 1 of the first embodiment explained above, one first light emitter 150′ and one light receiver 140 are mounted side by side on the substrate 160 (the sensor substrate). In the configuration of the sensor section 80 according to this configuration example 2, a plurality of light emitters (in this configuration example, two light emitters, i.e., a first light emitter 350 and a second light emitter 380) and one light receiver 340 are provided. The first light emitter 350 and the second light emitter 380 have a configuration same as the configuration of the first light emitter 150′ of the configuration example 1. Therefore, detailed explanation of the configuration is omitted. However, dome-type lenses 351 and 381 functioning as condensing members are provided respectively on the first light emitter 350 and the second light emitter 380.

The first light emitter 350, the second light emitter 380, and the light receiver 340 are mounted side by side in a row on a substrate 360 along a given direction in the order of the first light emitter 350, the light receiver 340, and the second light emitter 380. Specifically, the first light emitter 350, the light receiver 340, and the second light emitter 380 are disposed such that, in plan view of the light receiver 340 viewed from the Z-axis direction, a center P1 of the first light emitter 350, a center P2 of the second light emitter 380, and a center G of the light receiver 340 are located on one straight line (in this configuration, disposed along substantially the X-axis direction). Note that the first light emitter 350, the light receiver 340, and the second light emitter 380 are desirably disposed such that the distance between an outer peripheral side 350b of the first light emitter 350 on the light receiver 340 side and an outer peripheral side 340a of the light receiver 340 on the first light emitter 350 side and the distance between an outer peripheral side 380a of the second light emitter 380 on the light receiver 340 side and an outer peripheral side 340b of the light receiver 340 on the second light emitter 380 side are generally the same. The first wall section 70 is provided between the first light emitter 350 and the light receiver 340. A second wall section 70b is provided between the second light emitter 380 and the light receiver 340.

The light receiver 340 located between the first light emitter 350 and the second light emitter 380 has a configuration same as the configuration of the light receiver 140 of the configuration example 1 explained above. Therefore, detailed explanation of the configuration is omitted. However, an angle limiting filter 370 having a configuration same as the configuration of the angle limiting filter of the light receiver 140 is provided on the light receiver 340. The angle limiting filter 370 includes a plurality of first convex sections 347 arranged side by side along the first imaginary straight line Q1 (in this configuration, substantially the X-axis direction), which connects the center P1 of the first light emitter 350, the center P2 of the second light emitter 380, and the center G of the light receiver 340, in plan view of the light receiver 340 viewed from the Z-axis direction and projecting from the upper surface (the light receiving surface) of the light receiving element configuring the light receiver 340. As in the configuration example 1, each of the first convex sections 347 is formed in a plate wall shape extending along the first direction. In this configuration example 2, the heights of the respective first convex sections 347 from the upper surface of the light receiving element are set substantially the same. Note that the height of the first wall section 70 and the second wall section 70b from the substrate 360 is desirably larger than the height of the first convex sections 347 from the substrate 360.

With the sensor section 80 of the configuration example 2, lights are emitted from the plurality of light emitters (the first light emitter 350 and the second light emitter 380). Therefore, it is possible to make more intense light incident on the light receiver 340. It is possible to perform incidence limitation for an incident direction (an incident angle) of incident light on the light receiver 340. Consequently, it is possible to further improve detection accuracy of the biological-information measuring module.

By adopting such disposition, the optical path length between the first light emitter 350 and the light receiver 340 and the optical path length between the second light emitter 380 and the light receiver 340 are substantially the same. Lights emitted from the first light emitter 350 and the second light emitter 380 are substantially simultaneously made incident on the light receiver 340. Therefore, it is possible to improve an S/N ratio. That is, it is possible to improve measurement accuracy of the biological-information measuring device.

### Configuration Example 3

A sensor section 80a according to a configuration example 3 is explained with reference to FIG. 10B. In the configuration example 2, the configuration is explained in which the first wall section 70 and the second wall section 70b having the plate wall shape extended in one direction are provided. In the sensor section 80a of the configuration example 3, a ring-like wall section 71 surrounding an outer circumference 340c of the light receiver 340 is provided. In the sensor section 80a, the ring-like wall section 71 is different from the wall sections of the configuration example 2. The other components are the same. Therefore, the other components are denoted by reference numerals and signs same as those in the configuration example 2 and explanation of the components is omitted.

With the sensor section 80a according to the configuration example 3 including such a ring-like wall section 71, it is possible to attain effects same as the effects of the configuration example 2.

Note that the ring-like wall section 71 according to this configuration example 3 can be applied instead of the first wall section 70 in the configuration example 1.

### Configuration Example 4

A sensor section 90 according to a configuration example 4 is explained with reference to FIG. 11A. Compared with the sensor section 80 according to the configuration example 2, the configuration of the sensor section 90 according to the configuration example 4 is different in the disposition of the first light emitter 350 and the second light emitter 380 with respect to a light receiver 341. According to the difference, the configuration of an angle limiting filter 370b provided on the light receiver 341 is different. In the explanation of this configuration example 4, components different from the components of the sensor section 80 according to the configuration example 2 are mainly explained. Components same as the components of the sensor section 80 are denoted by the same reference numerals and signs and explanation of the components is sometimes omitted.

As in the configuration example 2, dome-type lenses 351 and 381 functioning as condensing members are respectively provided on the first light emitter 350 and the second light emitter 380 in the sensor section 90 according to this configuration example 4.

The first light emitter 350 has a gap between the first light emitter 350 and the light receiver 341. The first light emitter 350 and the light receiver 341 are mounted side by side in the first direction (the X-axis direction) on the substrate 360. The second light emitter 380 is present in a −X, +Y region and has a gap between the second light emitter 380 and the light receiver 341. The second light emitter 380 and the light receiver 341 are mounted side by side in the second direction crossing the first direction on the substrate 360. Specifically, the first light emitter 350 and the light receiver 341 are disposed such that the center P1 of the first light emitter 350 and the center G of the light receiver 341 are located on the first imaginary straight line Q1 along the first direction in plan view of the light receiver 341 viewed from the Z-axis direction. Similarly, the second light emitter 380 and the light receiver 341 are disposed such that the center P2 of the second light emitter 380 and the center G of the light receiver 341 are located on a second imaginary straight line Q2 along the second direction crossing the first direction (in this example, an axial direction obtained by rotating a −X axis approximately 45 degrees around a Z axis toward the Y-axis direction) in the same plan view. Note that the second direction is not limited to the tilt of 45 degrees of this example and may be set at any angle.

A first wall section 70a is provided between the first light emitter 350 and the light receiver 341. A second wall section 70b is provided between the second light emitter 380 and the light receiver 341. Note that the first wall section 70a has front and rear wall surfaces along the outer peripheral side 350b of the first light emitter 350 and is provided in a plate wall shape extending in the Y-axis direction. The second wall section 70b has front and rear wall surfaces along the outer peripheral side 380a of the second light emitter 380 and is provided in a plate wall shape extending in a direction substantially orthogonal to the second imaginary straight line Q2.

The light receiver 341 has a configuration same as the configuration of the light receiver 140 of the configuration example 1. Therefore, detailed explanation of the configuration is omitted. However, an angle limiting filter 370b having a configuration same as the configuration of the angle limiting filter of the light receiver 140 is provided on the light receiver 341. The angle limiting filter 370b includes a plurality of first convex sections 347a and a plurality of second convex sections 347b projecting from the upper surface (the light receiving surface) of the light receiving element configuring the light receiver 341. Specifically, the first convex sections 347a are arranged side by side along the first imaginary straight line Q1, which connects the center P1 of the first light emitter 350 and the center G of the light receiver 341, in plan view of the light receiver 341 viewed from the Z-axis direction and are provided in a plate wall shape extending in the extending direction (the first direction) of the first imaginary straight line Q1. The second convex sections 347b are arranged side by side along the second imaginary straight line Q2, which connects the center P2 of the second light emitter 380 and the center G of the light receiver 341, and are provided in a plate wall shape extending in the extending direction (the second direction) of the second imaginary straight line Q2. In other words, on the light receiver 341, a plurality of convex sections (in this example, the first convex sections 347a and the second convex sections 347b) parallel to one another toward a plurality of light emitters (in this example, the first light emitter 350 and the second light emitter 380) are disposed. The first convex sections 347a and the second convex sections 347b are connected in positions where the convex sections corresponding to one another respectively cross one another on the light receiver 341. Like the first convex sections 247 in the configuration example 1, the first convex sections 347a and the second convex sections 347b can be configured by alternately stacking, as functional layers, for example, a conductive layer such as an aluminum (light reflecting substance) wiring layer and a conductive plug layer such as a tungsten (light absorbing substance) plug.

In this configuration example 4, the heights of the first convex sections 374a and the second convex sections 347b from the upper surface of the light receiving element are set substantially the same. Note that the height of the first wall section 70a and the second wall section 70b from the substrate 360 is desirably larger than the height of the first convex sections 347a and the second convex sections 347b from the substrate 360. Since the first wall section 70a and the second wall section 70b are provided in this way, it is possible to prevent lights emitted from the first light emitter 350 and the second light emitter 380 from directly reaching (being made incident on) the light receiver 340. Consequently, it is possible to make light with fewer noise components incident on the light receiver 340. It is possible to further improve measurement accuracy of the biological-information measuring module.

With the sensor section 90 according to this configuration example 4, the first convex sections 347a and the second convex sections 347b are provided in parallel along the directions (the first direction and the second direction) from the first light emitter 350 and the second light emitter 380 to the light receiver 341. Therefore, it is possible to efficiently make lights emitted from the first light emitter 350 and the second light emitter 380, which are disposed in the respective positions, in the direction of the light receiver 341 incident on the light receiver 341 without blocking the lights. It is possible to perform incidence limitation for an incident direction (an incident angle) of incident light made incident on the light receiver 341 from a direction crossing the directions (the first direction and the second direction) from the first light emitter 350 and the second light emitter 380 to the light receiver 341. Since lights are emitted from the plurality of light emitters (the first light emitter 350 and the second light emitter 380), it is possible to make more intense light incident on the light receiver 341. Consequently, it is possible to further improve detection accuracy of the biological-information measuring module (the sensor section 90).

### Configuration Example 5

A sensor section 100 according to a configuration example 5 is explained with reference to FIG. 11B. In the configuration of the sensor section 100 according to this configuration example 5, compared with the sensor section 80 according to the configuration example 4, three light emitters are provided by adding one light emitter. The first light emitter 350, the second light emitter 380, and a third light emitter 390 are disposed on the outer side of an outer periphery 342a of a light receiver 342. According to the increase of the light emitters, the configuration of an angle limiting filter 370c provided on the light receiver 342 is different. In the explanation of this configuration example 5, components different from the components of the sensor section 90 according to the configuration example 4 are mainly explained. Components same as the components of the sensor section 90 are denoted by the same reference numerals and signs and explanation of the components is sometimes omitted.

First, the first light emitter 350, the second light emitter 380, and the third light emitter 390 are explained. As in the configuration example 4 explained above, dome-type lenses 351, 381, and 391 functioning as condensing members are respectively provided on the first light emitter 350, the second light emitter 380, and the third light emitter 390 in the sensor section 100.

The first light emitter 350 has a gap between the first light emitter 350 and the light receiver 342. The first light emitter 350 and the light receiver 342 are mounted side by side in the first direction (the X-axis direction) on the substrate 360. The second light emitter 380 is present in a −X, +Y region and has a gap between the second light emitter 380 and the light receiver 342. The second light emitter 380 and the light receiver 342 are mounted side by side in the second direction crossing the first direction on the substrate 360. The third light emitter 390 is present in a −X, −Y region and has a gap between the third light emitter 390 and the light receiver 342. The third light emitter 390 and the light receiver 342 are mounted side by side in a third direction crossing the first direction and the second direction on the substrate 360.

Specifically, the first light emitter 350 and the light receiver 342 are disposed such that the center P1 of the first light emitter 350 and the center G of the light receiver 342 are located on the first imaginary straight line Q1 along the first direction (the X-axis direction) in plan view of the light receiver 342 viewed from the Z-axis direction. Similarly, the second light emitter 380 and the light receiver 342 are disposed such that the center P2 of the second light emitter 380 and the center G of the light receiver 342 are located on the second imaginary straight line Q2 along the second direction crossing the first direction (in this example, the axial direction obtained by rotating the −X axis approximately 45 degrees around the Z axis toward the Y-axis direction) in the same plan view. Note that the second direction is not limited to the tilt of 45 degrees of this example and may be set at any angle. Similarly, the third light emitter 390 and the light receiver 342 are disposed such that a center P3 of the third light emitter 390 and the center G of the light receiver 342 are located on a third imaginary straight line Q3 along the third direction crossing the first direction (in this example, an axial direction obtained by rotating the −X axis approximately 45 degrees around the Z axis toward a −Y-axis direction) in the same plan view. Note that, as the third direction in this example, a direction tilting 45 degrees from the −X axis, in other words, a direction substantially orthogonal to the second imaginary straight line Q2 is illustrated. However, the third direction is not limited to this and may be set at any angle.

Between the first light emitter 350 and the light receiver 342, the first wall section 70a is provided to be separated from the first light emitter 350 and the light receiver 342. Between the second light emitter 380 and the light receiver 342, the second wall section 70b is provided to be separated from the second light emitter 380 and the light receiver 342. Between the third light emitter 390 and the light receiver 342, a third wall section 70c is provided to be separated from the third light emitter 390 and the light receiver 342. Note that the first wall section 70a has front and rear wall surfaces along the outer peripheral side 350b of the first light emitter 350 and is provided in a plate wall shape extending in the Y-axis direction. The second wall section 70b has front and rear wall surfaces along the outer peripheral side 380a of the second light emitter 380 and is provided in a plate wall shape extending in the direction substantially orthogonal to the second imaginary straight line Q2. The third wall section 70c has front and rear wall surfaces along an outer peripheral side 390a of the third light emitter 390 and is provided in a plate wall shape extending in a direction substantially orthogonal to the third imaginary straight line Q3.

The light receiver 342 has a configuration same as the configuration of the light receiver 140 of the configuration example 1. Therefore, detailed explanation of the configuration is omitted. However, an angle limiting filter 370c having a configuration same as the configuration of the angle limiting filter of the light receiver 140 is provided on the light receiver 342. The angle limiting filter 370c includes the plurality of first convex sections 347a, the plurality of second convex sections 347b, and a plurality of third convex sections 347c projecting from an upper surface (a light receiving surface) of a light receiving element configuration the light receiver 342. Specifically, the first convex sections 347a are arranged side by side along the first imaginary straight line Q1, which connects the center P1 of the first light emitter 350 and the center G of the light receiver 342, in plan view of the light receiver 342 viewed from the Z-axis direction and are provided in a plate wall shape extending in the extending direction (the first direction) of the first imaginary straight line Q1. The second convex sections 347b are arranged side by side along the second imaginary straight line Q2, which connects the center P2 of the second light emitter 380 and the center G of the light receiver 342, and are provided in a plate wall shape extending in the extending direction (the second direction) of the second imaginary straight line Q2. The third convex sections 347c are arranged side by side along the third imaginary line Q3, which connects the center P3 of the third light emitter 390 and the center G of the light receiver 342, and are provided in a plate wall shape extending in the extending direction (the third direction) of the third imaginary straight line Q3. In other words, on the light receiver 342, a plurality of convex sections (in this example, the first convex sections 347a, the second convex sections 347b, and the third convex sections 347c) parallel to one another toward a plurality of light emitters (in this example, the first light emitter 350, the second light emitter 380, and the third light emitter 390) are disposed.

The first convex sections 347a and the second convex sections 347b, the first convex sections 347a and the third convex sections 347c, and the second convex sections 347b and the third convex sections 347c are respectively connected in positions where the convex sections corresponding to one another respectively cross one another on the light receiver 342. In this configuration example 5, the heights of the first convex sections 347a, the second convex sections 347b, and the third convex sections 347c from the upper surface of the light receiving element are set substantially the same. Note that the height of the first wall section 70a, the second wall section 70b, and the third wall section 70c from the substrate 360 is desirably larger than the height of the first convex sections 347a, the second convex sections 347b, and the third convex sections 347c from the substrate 360.

With the sensor section 100 according to this configuration example 5, the first convex sections 347a, the second convex sections 347b, and the third convex sections 347c are provided in parallel along the directions (the first direction, the second direction, and the third direction) from the first light emitter 350, the second light emitter 380, and the third light emitter 390 to the light receiver 342. Consequently, it is possible to efficiently make lights emitted from the first light emitter 350, the second light emitter 380, and the third light emitter 390, which are disposed in the respective positions, in the direction of the light receiver 342 incident on the light receiver 342 without blocking the lights. It is possible to perform incidence limitation for an incident direction (an incident angle) of incident light made incident on the light receiver 342 from a direction crossing the directions (the first direction, the second direction, and the third direction) from the first light emitter 350, the second light emitter 380, and the third light emitter 390 to the light receiver 342. In addition, since lights are emitted from the many light emitters (the first light emitter 350, the second light emitter 380, and the third light emitter 390), it is possible to make more intense light incident on the light receiver 342. Consequently, it is possible to further improve detection accuracy of the biological-information measuring module (the sensor section 100).

### Configuration Example 6

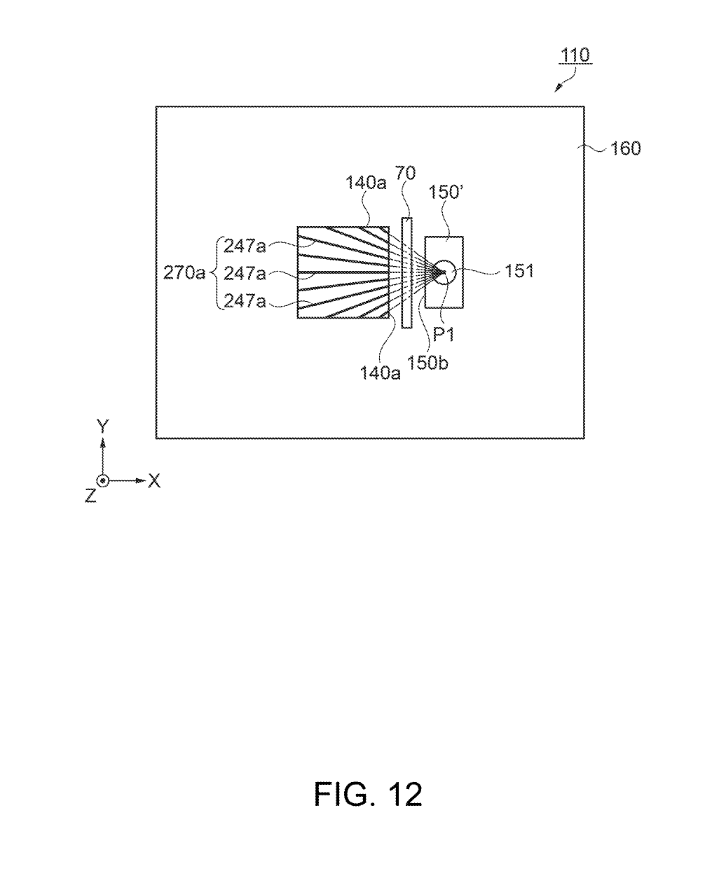

A sensor section 110 according to a configuration example 6 is explained with reference to FIG. 12. In the configuration of the sensor section 110 according to the configuration example 6, as in the sensor section 40 according to the configuration example 1, one first light emitter 150′ and one light receiver 140a are mounted side by side on the substrate 160 (the sensor substrate). In the sensor section 110 according to the configuration example 6, compared with the sensor section 40 according to the configuration example 1, the configuration of an angle limiting filter 270a provided in the light receiver 140a is different. The other components such as the light receiver 140a and the first light emitter 150′ excluding the angle limiting filter 270a are the same as the components of the sensor section 40 of the configuration example 1. Therefore, detailed explanation of the components is omitted.

The sensor section 110 of the configuration example 6 includes the light receiver 140a, the first light emitter 150′, and the first wall section 70 provided between the light receiver 140a and the first light emitter 150′ to be separated from the light receiver 140a and the first light emitter 150′. The light receiver 140a and the first light emitter 150′ are arranged side by side at a predetermined interval and mounted on the substrate 160 (the sensor substrate) functioning as the supporter.

The light emitter 140a in the configuration example 6 includes the angle limiting filter (a filter section) 270a in which a plurality of convex sections 247a arranged radially with the center P1 of the first light emitter 150′ set as a reference point (a start point) in plan view of the light receiver 140a viewed from the Z-axis direction and projecting from the light receiving surface are formed. In other words, the respective plurality of convex sections 247a extend toward the dome-type lens 151 functioning as the condensing member of the first light emitter 150′.

With the sensor section 110 according to the configuration example 6, in the light receiver 140a, the angle limiting filter (the filter section) 270a including the plurality of convex sections 247a arranged radially with the center P1 of the first light emitter 150′ set as the reference point (the start point) in plan view and projecting from the light receiving surface is provided. With the angle limiting filter (the filter section) 270a provided in this way, it is possible to more efficiently make light emitted from the first light emitter 150′ incident on the light receiver 140a. Further, it is possible to suppress light from a direction crossing the convex sections 247a arranged radially.

By providing the angle limiting filter (the filter section) 270a on the light receiver 140a, it is possible to configure a smaller biological-information measuring module without changing a plane size, that is, without increasing the size of the biological-information measuring module.

### Modifications of the Angle Limiting Filter

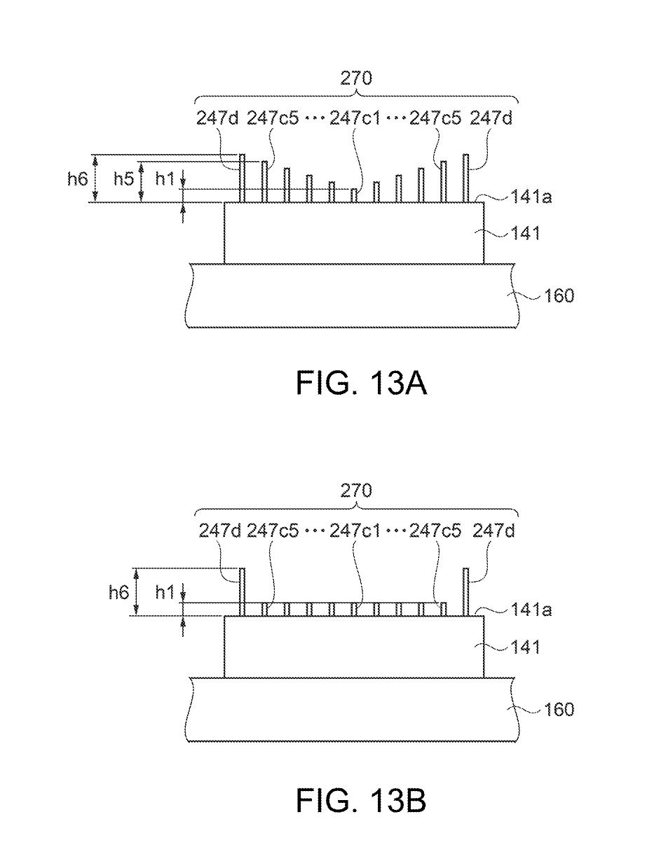

Modifications of the angle limiting filter functioning as the filter section are explained with reference to FIGS. 13A and 13B.

### Modification 1

FIG. 13A is a diagram showing a modification 1 of the angle limiting filter functioning as the filter section and is a sectional view equivalent to A-A view of FIG. 8A.

In FIG. 13A, the angle limiting filter 270 functioning as the filter section according to this modification 1 includes a plurality of first convex sections 247c1 to 247c5 and 247d. As the first convex sections 247c1 to 247c5 and 247d, the first convex sections 247c2 to 247c5 are disposed side by side on both sides of the first convex section 247c1 located in the center and the first convex sections 247d functioning as end portion convex sections are disposed on the outer sides of the first convex sections 247c2 to 247c5. The first convex section 247c1 to the first convex sections 247d functioning as the end portion convex sections are configured such that heights from the upper surface (the light receiving surface) 141a of the semiconductor substrate 141 sequentially increase. In other words, height h6 from the upper surface (the light receiving surface) 141a of the first convex sections 247d functioning as the end portion convex sections located at end portions of the angle limiting filter 270 in a direction in which the first convex sections 247c1 to 247c5 and 247d are arranged among the plurality of first convex sections 247c1 to 247c5 and 247d is set larger than heights h1 to h5 from the upper surface (the light receiving surface) 141a of the other first convex sections 247c1 to 247c5. Note that the other components of the plurality of first convex sections 247c1 to 247c5 and 247d are the same as the components in the configuration example 1. Therefore, explanation of the components is omitted.

### Modification 2

FIG. 13B is a diagram showing a modification 2 of the angle limiting filter functioning as the filter section and is a sectional view equivalent to the A-A view of FIG. 8A.

In FIG. 13B, the angle limiting filter 270 functioning as the filter section according to this modification 2 includes the plurality of first convex sections 247c1 to 247c5 and 247d. As the first convex sections 247c1 to 247c5 and 247d, the first convex sections 247c2 to 247c5 are disposed side by side on both sides of the first convex section 247c1 located in the center and the first convex sections 247d functioning as the end portion convex sections are disposed on the outer sides of the first convex sections 247c2 to 247c5. The height h1 from the upper surface (the light receiving surface) 141a is substantially the same in the first convex section 247c1 to the first convex sections 247c5. The first convex sections 247d functioning as the end portion convex section are configured such that the height h6 from the upper surface (the light receiving surface) 141a is larger than the height h1 of the first convex section 247c1 to the first convex sections 247c5. In other words, the height h6 from the upper surface (the light receiving surface) 141a of the first convex sections 247d functioning as the end portion convex sections located at the end portions of the angle limiting filter 270 in the direction in which the first convex sections 247c1 to 247c5 and 247d are arranged among the plurality of first convex sections 247c1 to 247c5 and 247d is set larger than the height h1 from the upper surface (the light receiving surface) 141a of the other first convex sections 247c1 to 247c5. Note that the other components of the plurality of first convex sections 247c1 to 247c5 and 247d are the same as the components in the configuration example 1. Therefore, explanation of the components is omitted.

With the angle limiting filter 270 functioning as the filter section having the configurations of the modification 1 and the modification 2, with the high first convex sections 247d functioning as the end portion convex sections located at the end portions of the filter section, it is possible to efficiently perform incidence limitation for an incident direction (an incident angle) of incident light on the light receiver 140 from a direction crossing a direction (e.g., the first direction) from the light emitter such as the first light emitter 150′ to the light receiver 140. It is possible to efficiently make light traveling from the light emitter such as the first light emitter 150′ to the light receiver 140 incident on the light receiver 140.

### Fourth Embodiment

A fourth embodiment of the invention is explained with reference to the drawings.

A biological-information measuring device functioning as an electronic device according to the fourth embodiment is, as in the first embodiment, a heart-rate monitoring device worn on an organism (e.g., a human body), biological information of which is measured, to measure biological information such as a pulse (a heart rate). Note that in the figures referred to below, dimensions and ratios of components are sometimes shown different from those of actual components as appropriate in order to show the components in recognizable sizes on the figures. In embodiments 4 to 7 explained below, the configuration of the pulse-wave measuring module explained in the first embodiment is adopted. For example, the structure of the optical filter 143 can be applied in the same manner.

First, before explaining a heart-rate monitoring device 1010 functioning as the electronic device (the biological-information measuring device) according to the fourth embodiment, a related art of the heart-rate monitoring device functioning as the biological-information measuring device according to the fourth embodiment is explained with reference to FIG. 14.

FIG. 14 is a sectional view showing the heart-rate monitoring device 1010 functioning as the biological-information measuring device of the related art that measures physiological parameters (biological information) of a user (a test object) 1000 (in the figure, indicating an arm of the user) wearing the heart-rate monitoring device. The heart-rate monitoring device 1010 includes a sensor 1012 that measures a heart rate serving as at least one physiological parameter of the user 1000 and a case 1014 that houses the sensor 1012. The heart-rate monitoring device 1010 is worn on an arm 1001 of the user 1000 by a fixing section 1016 (e.g., a band).

The sensor 1012 is a heart-rate monitoring sensor including a light emitting element 1121 functioning as a light emitter and a light receiving element 1122 functioning as a light receiver, which are two sensor elements, to measure or monitor a heart rate. However, the sensor 1012 may be a sensor that measures one or more physiological parameters (e.g., a heart rate, a blood pressure, a tidal volume, skin conductivity, and skin humidity). When the case 1014 includes a band-type housing, the sensor 1012 can be used as a wristwatch-type monitoring device used in, for example, sports. Note that the shape of the case 1014 only has to be a shape for mainly enabling the sensor 1012 to be held in a desired position of the user 1000. The case 1014 may be able to optionally further house components such as a battery, a processing unit, a display, and a user interface.

The biological-information measuring device of the related art is a heart-rate monitoring device 1010 for monitoring a heart rate of the user. The sensor 1012 is an optical sensor including the light emitting element 1121 and the light receiving element 1122. An optical heart rate monitor including the optical sensor relies on the light emitting element 1121 (usually, an LED is used) functioning as a light source for radiating light on skin. A part of the light radiated on the skin from the light emitting element 1121 is absorbed by blood flowing in a blood vessel under the skin. However, the remaining light is reflected by the blood vessel to the outside of the skin. The reflected light is captured by the light receiving element 1122 (usually, a photodiode is used). A light reception signal from the light receiving element 1122 is a signal including information equivalent to an amount of blood flowing in the blood vessel. The amount of blood flowing in the blood vessel changes according to the pulsation of the heart. In this way, the signal on the light receiving element 1122 changes according to the beat of the heart. That is, the change in the signal of the light receiving element 1122 is equivalent to a pulse of a heart rate. The number of beats (i.e., a heart rate) in one minute of the heart is obtained by counting the number of pulses per unit time (e.g., ten seconds).

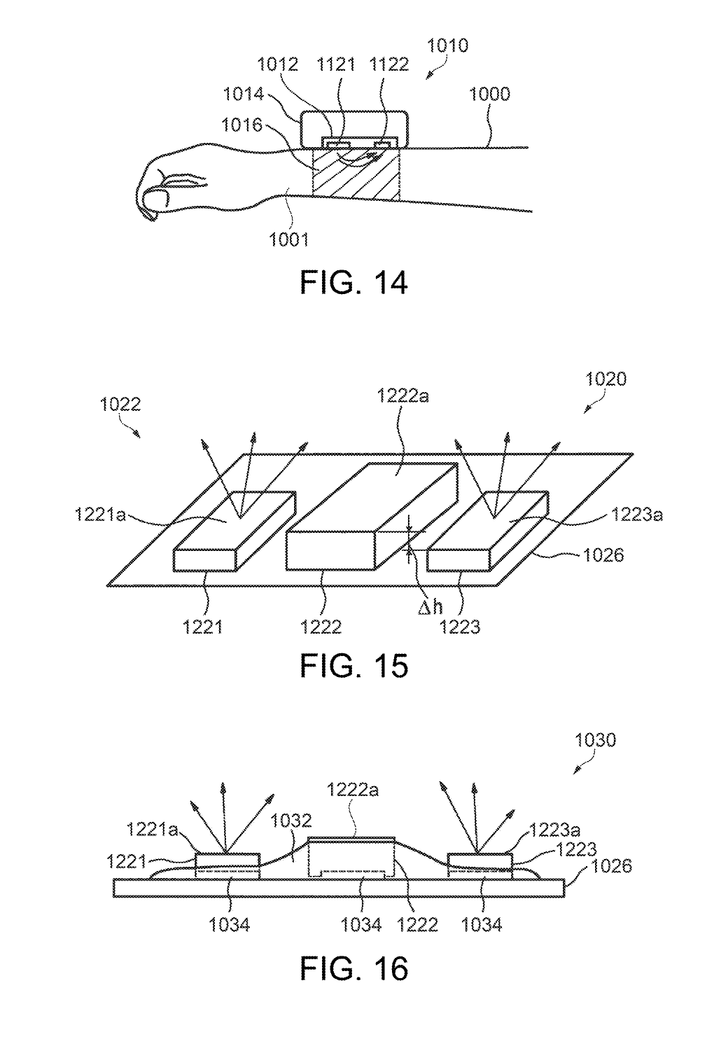

A heart-rate monitoring device 1020 functioning as the electronic device (the biological-information measuring device) according to the fourth embodiment is explained with reference to FIG. 15. FIG. 15 is a perspective view showing the heart-rate monitoring device functioning as the biological-information measuring device according to the fourth embodiment. Although not shown in FIG. 15, as in the first embodiment, the heart-rate monitoring device 1020 functioning as the biological-information measuring device according to the fourth embodiment is worn on the arm of the user by a fixing section such as a band.

In the heart-rate monitoring device 1020 functioning as the biological-information measuring device according to the fourth embodiment, light emitting elements 1221 and 1223 functioning as a plurality of (in this example, two) light emitters and a light receiving element 1222 functioning as one light receiver are disposed side by side in a row. Specifically, the heart-rate monitoring device 1020 includes a sensor 1022 including at least two sensor elements (in this example, as three sensor elements, the two light emitting elements 1221 and 1223 functioning as a first light emitting element and a second light emitting element and the light receiving element 1222 functioning as a light receiving element are used). Note that, although not shown in the figure, the wall section 70 (see FIGS. 5A and 5B) having a configuration same as the configuration example explained above is desirably provided between the light receiving element 1222 and the light emitting element 1221 and between the light receiving element 1222 and the light emitting element 1223.

The light receiving element 1222 functioning as the light receiver is disposed between the two light emitting elements 1221 and 1223 functioning as the first light emitter and the second light emitter. The two light emitting elements 1221 and 1223 functioning as the first light emitter and the second light emitter are disposed in symmetrical positions with respect to an imaginary line passing the center of the light receiving element 1222 functioning as the light receiver. By disposing the light emitting elements 1221 and 1223 and the light receiving element 1222 in this way, a dead space decreases. It is possible to attain space saving. Lights from the first light emitter and the second light emitter present in the symmetrical positions are collected in the light receiver. It is possible to perform more accurate detection.

The sensor elements detect a sensor signal. The sensor 1022 includes an optical sensor including the light emitting elements 1221 and 1223, in which two LEDs are used, for emitting lights to the skin of the user and at least one light receiving element 1222 (photodiode) for receiving light reflected from the skin. Further, the heart-rate monitoring device 1020 includes a case or a housing (not shown in the figure). The case or the housing may be similar to or the same as the case 1014 shown in FIG. 14 or may be similar to or the same as the case section 30 in the first embodiment.

The sensor 1022 is carried on the entire surface of a carrier (a substrate) 1026. A component including the carrier (the substrate) 1026 and the sensor 1022 carried on the carrier (the substrate) 1026 is equivalent to a pulse-wave measuring module. The same applies in fifth to seventh embodiments explained below. Lights emitted from the light emitting elements 1221 and 1223 are reflected without being absorbed by the skin and the like and can directly reach the light receiving element 1222. In the heart-rate monitoring device 1020, the distance between the carrier 1026 and upper surfaces 1221a and 1223a of the light emitting elements 1221 and 1223 is smaller than the distance between the carrier 1026 and an upper surface 1222a of the light receiving element 1222. That is, a difference between the distance between the carrier 1026 and the upper surfaces 1221a and 1223a of the light emitting elements 1221 and 1223 and the distance between the carrier 1026 and the upper surface 1222a of the light receiving element 1222 is Ah. The light receiving element 1222 receives light from the upper surface 1222a, which is the uppermost layer of the light receiving element 1222. With these components, there is an effect that most of the lights emitted from the light emitting elements 1221 and 1223 travels to the skin and reflected light is directly made incident on the light receiving element 1222 without intervention of an air layer and the like. In other words, since the light receiving element 1222 is in contact with the skin, a gap is less easily formed between the upper surface (the light receiving surface) 1222a of the light receiving element 1222 and the skin. Consequently, it is possible to suppress light such as external light, which is a noise source, from being made incident on the upper surface 1222a. Lights from the light emitting elements 1221 and 1223 not passing through the skin, for example, lights directly made incident on the light receiving elements 1222 from the light emitting elements 1221 and 1223 cannot reach the upper surface 1222a of the light receiving element 1222.

### Fifth Embodiment

A heart-rate monitoring device 1030 functioning as an electronic device (a biological-information measuring device) according to a fifth embodiment is explained with reference to FIG. 16. FIG. 16 is a side view showing the heart-rate monitoring device functioning as the biological-information measuring device according to the fifth embodiment. Note that, although not shown in FIG. 16, as in the first embodiment, the heart-rate monitoring device 1030 functioning as the biological-information measuring device according to the fifth embodiment is worn on an arm of a user by a fixing section such as a band.

As shown in FIG. 16, electric connection terminals 1034 of the light emitting elements 1221 and 1223 functioning as the light emitters and the light receiving element 1222 functioning as the light receiver desirably have to be covered with an insulative material (e.g., epoxy resin) 1032 for protection of electric elements. The insulative material 1032 can be configured not to cover the light emitting elements 1221 and 1223 and the light receiving element 1222. Specifically, a region between the light emitting element 1221 and the light receiving element 1222 and a region between the light emitting element 1223 and the light receiving element 1222 can be filled with the insulative material 1032. In other words, at least the upper surface 1222a of the light receiving element 1222 and the upper surfaces 1221a and 1223a of the light emitting elements 1221 and 1223 can be configured not to be covered with the insulative material 1032. By adopting such a configuration, it is possible to suppress interference by air gaps between the skin and the light emitting elements 1221 and 1223. Further, the insulative material 1032 may be configured to cover the upper surfaces 1221a and 1223a of the light emitting elements 1221 and 1223 and the upper surface 1222a of the light receiving element 1222. By adopting such a configuration, it is possible to protect the upper surface 1222a of the light receiving element 1222 in contact with the skin and the upper surfaces 1221a and 1223a of the light emitting elements 1221 and 1223. Therefore, it is possible to prevent damage to the upper surface 1222a of the light receiving element 1222 and the upper surfaces 1221a and 1223a of the light emitting elements 1221 and 1223. In this case, the insulative material 1032 can also be regarded as a protection film.

In the heart-rate monitoring device 1030 functioning as the biological-information measuring device according to the fifth embodiment, as a generally possible example, the insulative material 1032 including epoxy resin is provided. In FIG. 16, the insulative material 1032 is disposed not to cover the upper surfaces 1221a and 1223a of the light emitting elements 1221 and 1223 and protects the electric connection terminals 1034. Lights emitted from the light emitting elements 1221 and 1223 are indicated by arrows.

In this way, the insulative material 1032 is disposed as small as possible not to prevent the correct function of the heart-rate monitoring device 1030, whereby the electric connection terminals 1034 of the light emitting elements 1221 and 1223 and the light receiving element 1222 are protected. Consequently, the heart-rate monitoring device 1030 can be further improved. Note that, although not shown in the figure, it is more suitable that the wall section 70 (see FIGS. 5A and 5B) same as the configuration example is provided between the light receiving element 1222 and the light emitting element 1221 and between the light receiving element 1222 and the light emitting element 1223.

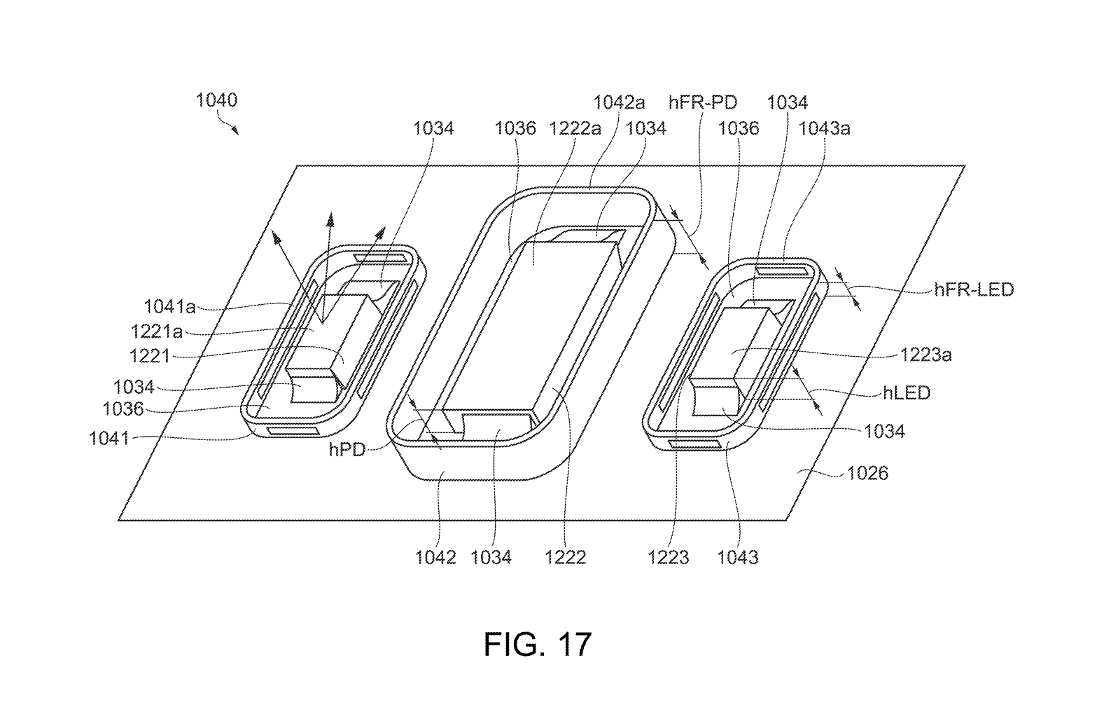

Note that it is more suitable to adopt, instead of the configuration in which the epoxy resin is injected in the fifth embodiment, a heart-rate monitoring device 1040 functioning as a biological-information measuring device according to a sixth embodiment shown in FIG. 17.

### Sixth Embodiment

A heart-rate monitoring device 1040 functioning as an electronic device (a biological-information measuring device) according to a sixth embodiment is explained with reference to FIG. 17. FIG. 17 is a perspective view showing the heart-rate monitoring device functioning as the biological-information measuring device according to the sixth embodiment. Note that, although not shown in FIG. 17, as in the first embodiment, the heart-rate monitoring device 1040 functioning as the biological-information measuring device according to the sixth embodiment is worn on an arm of a user by a fixing section such as a band.

The heart-rate monitoring device 1040 functioning as the biological-information measuring device according to the sixth embodiment includes a frame section 1042 surrounding a light receiver in a frame shape. Specifically, in the heart-rate monitoring device 1040, created frame sections 1041, 1042, and 1043 are disposed. The frame section 1042 is disposed around the light receiving element 1222 functioning as the light receiver. The frame sections 1041 and 1043 are disposed around the light emitting elements 1221 and 1223 functioning as the light emitters. Gaps 1036 between the frame sections 1041, 1042, and 1043 and the light emitting elements 1221 and 1223 and the light receiving element 1222 are formed. An insulative material (not shown in FIG. 17) is injected using the frame sections 1041, 1042, and 1043 as guides and covers the electric connection terminals 1034 of the light emitting elements 1221 and 1223 and the light receiving element 1222.

In an example explained in the sixth embodiment, the light emitting elements 1221 and 1223 and the light receiving element 1222 are surrounded by the respective frame sections 1041, 1042, and 1043. Note that, as another example, all of the frame sections 1041, 1042, and 1043 may be joined to one another. Alternatively, all sensor elements may be surrounded by an integral frame section. Note that the frame sections 1041, 1042, and 1043 can be used as light blocking walls (wall sections), which are an example of a light blocker. By using the frame sections 1041, 1042, and 1043 as the light blocking walls (the wall sections), it is possible to prevent lights emitted from the light emitting elements 1221 and 1223 from being directly entering the light receiving element 1222.

As an improvement for preventing the function of the heart-rate monitoring device 1040 from being affected, upper edges 1041a and 1043a of the frame sections 1041 and 1043 around the light emitting elements 1221 and 1223 are desirably lower than the upper surfaces 1221a and 1223a of the light emitting elements 1221 and 1223. In other words, a distance hFR−LED between the upper edges 1041a and 1043a of the individual frame sections 1041 and 1043 and the carrier 1026 is the same as or smaller than a distance hLED between the upper surfaces 1221a and 1223a of the light emitting elements 1221 and 1223 surrounded by the individual frame sections 1041 and 1043 and the carrier 1026 (hFR−LED≦hLED).

Desirably, a difference between the distance hLED between the upper surfaces 1221a and 1223a of the light emitting elements 1221 and 1223 and the carrier 1026 and the distance hFR−LED between the upper edges 1041a and 1043a of the frame sections 1041 and 1043 and the carrier 1026 is set in a range of 0.1 mm to 0.8 mm. Note that, more desirably, the difference between the distance hLED between the upper surfaces 1221a and 1223a of the light emitting elements 1221 and 1223 and the carrier 1026 and the distance hFR−LED between the upper edges 1041a and 1043a of the frame sections 1041 and 1043 and the carrier 1026 is set in a range of 0.2 mm to 0.5 mm.

The upper edge 1042a of the frame section 1042 is desirably higher than the upper surface 1222a of the light receiving element 1222 functioning as the light receiver. In other words, a distance hFR−PD between the upper edge 1042a of the frame section 1042 and the carrier 1026 is larger than a distance hPD between the upper surface 1222a of the light receiving element 1222 surrounded by the frame section 1042 and the carrier 1026 (hFRPD>hPD).

Desirably, a difference between the distance hPD between the upper surface 1222a of the light receiving element 1222 and the carrier 1026 and the distance hFR−PD between the upper edge 1042a of the frame section 1042 and the carrier 1026 is set in a range of 0 mm to 0.5 mm. Note that, more desirably, the difference between the distance hPD between the upper surface 1222a of the light receiving element 1222 and the carrier 1026 and the distance hFR−PD between the upper edge 1042a of the frame section 1042 and the carrier 1026 is set in a range of 0.1 mm to 0.2 mm. Consequently, the upper edge 1042a of the frame section 1042 and the skin of the test object come into contact with each other, whereby it is possible to prevent incidence of external light. Pressing is stabilized by the frame section 1042 and a contact state of the light receiving element 1222 and the skin of the test object is stable during measurement. Therefore, it is possible to stably detect the reflected light.

Further, the distance hFR−PD between the upper edge 1042a of the frame section 1042 and the carrier 1026 is larger than the distance hLED between the upper surfaces 1221a and 1223a of the light emitting elements 1221 and 1223 and the carrier 1026 (hFR−PD>hLED).

Note that, for example, when the light receiving element 1222 and the light emitting elements 1221 and 1223 are close to each other, only one frame wall may be present between the light receiving element 1222 and the light emitting elements 1221 and 1223. This is sometimes caused because of manufacturing easiness. When the one frame wall is a case, frame walls of frames coincide with each other in both of the light receiving element 1222 and the light emitting elements 1221 and 1223. This means that the frame walls of the light emitting elements 1221 and 1223 are higher. Specifically, the frame walls on a side where the light receiving element 1222 is present in the frame sections 1041 and 1043 surrounding the light emitting elements 1221 and 1223 are high. The other frame walls are lower than the upper surfaces 1221a and 1223a of the light emitting elements 1221 and 1223.

Further, instead of the frame sections 1041, 1042, and 1043, a first wall section may be provided between the light receiving element 1222 and the light emitting element 1221 or the light emitting element 1223 and a second wall section may be provided on the outer side of the light emitting elements 1221 and 1223, that is, on the opposite side of the first wall section with respect to the light receiving element 1222.

In such a configuration, the distance between the carrier 1026 and the upper surface of the first wall section may be set larger than the distance between the carrier 1026 and the upper surface of the second wall section. By adopting such a configuration, compared with when the light emitting elements and the light receiving element are surrounded as shown in FIG. 17, it is possible to realize the function of the frames with fewer members.

Note that, by using the frame sections 1041 and 1043 and the frame sections 1042 as in the sixth embodiment, it is possible to prevent an injected insulative material such as epoxy resin from flowing out. Creating an additional structure to partition the insulative material such as the epoxy resin in this way is an option for enabling high mass productivity. Note that the frame sections 1041 and 1043 and the frame section 1042 may be formed of a material same as the material of the carrier 1026. For example, the frames may be formed by injection molding using epoxy-based resin or polycarbonate-based resin.

As explained above, the insulative material 1032 (see FIG. 16) protects the electric connection terminals 1034 of the sensor elements (the light emitting elements 1221 and 1223 and the light receiving element 1222). However, the electric connection terminals 1034 have to be further in contact with additional electronic devices (e.g., a driver, a detection electronics, a processor, and a power supply), which are other element. This means that some electric connection to the additional electronic devices is present in the carrier 1026 (which may be a printed board (PCB)). The structure of the heart-rate monitoring device according to this embodiment can be applied to not only a measuring device of a heart rate but also a measuring device of a pulse wave and a pulse.

### Seventh Embodiment

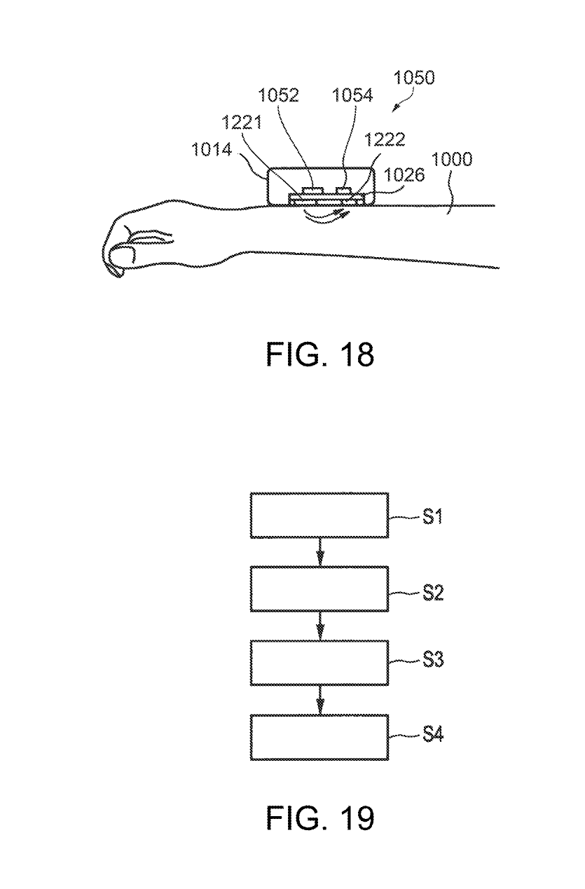

A heart-rate monitoring device 1050 functioning as a biological-information measuring device according to a seventh embodiment is explained with reference to FIG. 18. FIG. 18 is a sectional view showing the heart-rate monitoring device functioning as the biological-information measuring device according to the seventh embodiment. Note that, although not shown in FIG. 18, as in the first embodiment, the heart-rate monitoring device 1050 functioning as the biological-information measuring device according to the seventh embodiment is worn on an arm of a user by a fixing section such as a band.

The heart-rate monitoring device 1050 functioning as the biological-information measuring device according to the seventh embodiment includes the additional electronic devices (e.g., a processor 1052 and a driver 1054) explained above. An external electric connection terminal (not shown in the figure) is not disposed on the carrier 1026 on which the sensor elements (the light emitting element 1221 functioning as the light emitter and the light receiving element 1222 functioning as the light receiving element) are disposed. That is, the additional electronic devices are disposed on a carrier or a substrate different from a carrier or a substrate on which the sensor elements are disposed. By adopting such a configuration, it is possible to mount necessary additional electronic devices on the heart-rate monitoring device 1050 while maintaining satisfactory contact of the skin and the sensor elements (the light emitting element 1221 and the light receiving element 1222). For example, the external electric connection terminal can be disposed on a side surface of the carrier 1026.

As explained above, different kinds of sensors can be used in the biological-information measuring device according to the invention. For example, when the light receiving element 1222 is an electric sensor, two skin conductance electrodes (e.g., the sensor elements (the light emitting element 1221 and the light receiving element 1222 shown in FIG. 15)) set in contact with the skin of the user to measure the conductivity of the user are covered with the skin. Note that further two or more kinds of sensors can be used in the biological-information measuring device of this type. Further, the number of sensor elements may be any number.

A flowchart of a method of manufacturing a biological-information measuring device that measures proposed physiological parameters in the fourth to seventh embodiments is shown in FIG. 19.

In a first step S1, the sensor 1022 including at least two sensor elements (the light emitting element 1221 and the light receiving element 1222) for detecting a sensor signal is disposed on the carrier 1026. In a second step S2, an electric contact of the sensor elements is formed on the carrier 1026. In a third step S3, one or more frame sections 1041 and 1042 are formed on the carrier 1026 around the sensor 1022 and/or the respective sensor elements (the light emitting element 1221 and the light receiving element 1222). In a fourth step S4, the insulative material 1032 is injected and filled in regions that do not cover the upper surfaces 1221a and 1222a of the sensor elements (the light emitting element 1221 and the light receiving element 1222) provided on the carrier 1026 and are surrounded by the respective frame sections 1041 and 1042.

According to the fourth to seventh embodiments, there is proposed a method of achieving protection of the electric contact without adversely affecting the performance of the biological-information measuring device. The biological-information measuring device is formed by a method of keeping the performance of the sensors. For example, at least one of the frame sections 1041 and 1043 prevents the positions of the sensors with respect to the skin from shifting. Further, at least one of the frame sections 1041 and 1043 can be useful for preventing emitted direct light from being incident on the light receiving element 1222. Desirably, the height of the frame sections 1041 and 1043 around the light emitting elements 1221 and 1223 on a side facing the light receiving element 1222 has to be smaller than the height of the upper surfaces 1221a and 1223a of the light emitting elements 1221 and 1223. In addition, the frame section 1042 around the light receiving element 1222 may be higher than the upper surface 1222a of the light receiving element 1222.

In the electronic device (the biological-information measuring device) according to the fourth to seventh embodiments, effects same as the effects of the first embodiment can be obtained by mounting the sensor section 40 functioning as the pulse-wave measuring module explained in the first embodiment.

With the electronic device according to the sixth embodiment, the heart-rate monitoring device 1040 functioning as the electronic device includes the frame section 1042 surrounding, in a frame shape, the light receiving element 1222 functioning as the light receiver. The upper edge 1042a of the frame section 1042 is higher than the upper surface 1222a of the light receiving element 1222 functioning as the light receiver. Therefore, the upper edge 1042a of the frame section 1042 and the skin of the test object come into contact with each other, whereby it is possible to prevent incidence of external light. Pressing is stabilized by the frame section 1042 and a contact state of the light receiving element 1222 and the skin of the test object is stable during measurement. Therefore, it is possible to stably detect the reflected light.

### Eighth Embodiment

The biological-information measuring device according to the first to seventh embodiments may include various sensors such as a strain meter, a thermometer, a clinical thermometer, an acceleration sensor, a gyro sensor, a piezoelectric sensor, an atmospheric pressure sensor, a manometer, an electrochemical sensor, a GPS (Global Positioning System), and a vibrometer. By including these sensors, it is possible to derive information concerning a personal physiological state on the basis of data indicating one or one or more physiological parameters such as a beat, a pulse, a variation between pulsations, an EKG (ElectroKardiogram: electrocardiogram), an ECG (Electrocardiogram), a breathing rate, a skin temperature, a body temperature, a heat flow of a body, an electric skin reaction, a GSR (Galvanic skin reflex), an EMG (Electromyogram), an EEG (electroencephalogram), an EOG (Electrooculography), a blood pressure, a body fat, a hydration level, an activity level, a body motion, an oxygen consumption, glucose, a blood sugar level, a muscle mass, pressure on muscles, pressure on bones, ultraviolet ray absorption, a sleeping state, a physical condition, a stress state, and a posture (e.g., lying, upright, or sitting). Values obtained by the various sensors may be transmitted to a portable communication terminal such as a smart phone, a cellular phone, or a future phone or an information processing terminal such as a computer or a tablet computer to execute arithmetic processing of the physiological parameters in the portable communication terminal or the information processing terminal.

Before measuring biological information, the user inputs a profile of the user to the biological-information measuring device, the portable communication terminal, or the information processing terminal. Consequently, in order to maximize the possibility of establishing and maintaining a recommended healthy life style on the basis of the profile and a biological information measurement result, the user can receive provision of characteristic information peculiar to the user and environment information that need to be treated. Examples of the presented information include one kind or a plurality of kinds of information including exercise information such as an exercise type, exercise intensity, and an exercise time, meal information such as a meal time, an amount of meals, recommended intake food materials and intake menus, and intake food materials and intake menus that should be avoided, life support information such as a sleep time, depth of sleep, quality of sleep, a wakeup time, a bed time, a working time, stress information, a consumed calorie, an intake calorie, and a calorie balance, body information such as basal metabolism, a body fat amount, a body fat percentage, and a muscle mass, medication information, supplement intake information, and medical information.

Examples of the profile of the user input beforehand include one or a plurality of, for example, an age, a date of birth, sex, a hobby, an occupation category, a blood type, a sports history in the past, an activity level, meals, regularity of sleep, regularity of a bowel habit, situation adaptability, persistence, responsiveness, strength of reaction, a personality of the user such as characters, an independency level of the user, self-organization, self-management, sociability, a memory and an academic accomplishment ability, an awakening level of the user, attentiveness of the user including cognition speed, an avoidance ability for an attentiveness hindrance factor, and an awakening state and a self-control ability, an attention maintenance ability, weight, height, a blood pressure, a health state of the user, a diagnosis result by a doctor, a diagnosis date by the doctor, presence or absence of contact with the doctor and a health manager, drugs and supplements currently taken, presence or absence of allergies, an allergy history, a present allergy symptom, an opinion concerning a behavior related to health, a disease history of the user, a surgery history of the user, a family history, a social event such as a divorce or unemployment that required adjustment by an individual, an opinion concerning health priority of the user, a sense of value, an ability to change a behavior, an event considered to be a stress cause of life, a stress management method, a self-consciousness degree of the user, an empathy degree of the user, an authority transfer degree of the user, self-respect of the user, exercise of the user, a sleep state, a relaxed state, a present routine of everyday activities, a personality of an important person (e.g., a spouse, a friend, a colleague, or a superior) in the life of the user, and a perception of the user concerning whether a collision inhibiting a healthy life style or contributing to stress in a relation with the important person is present.

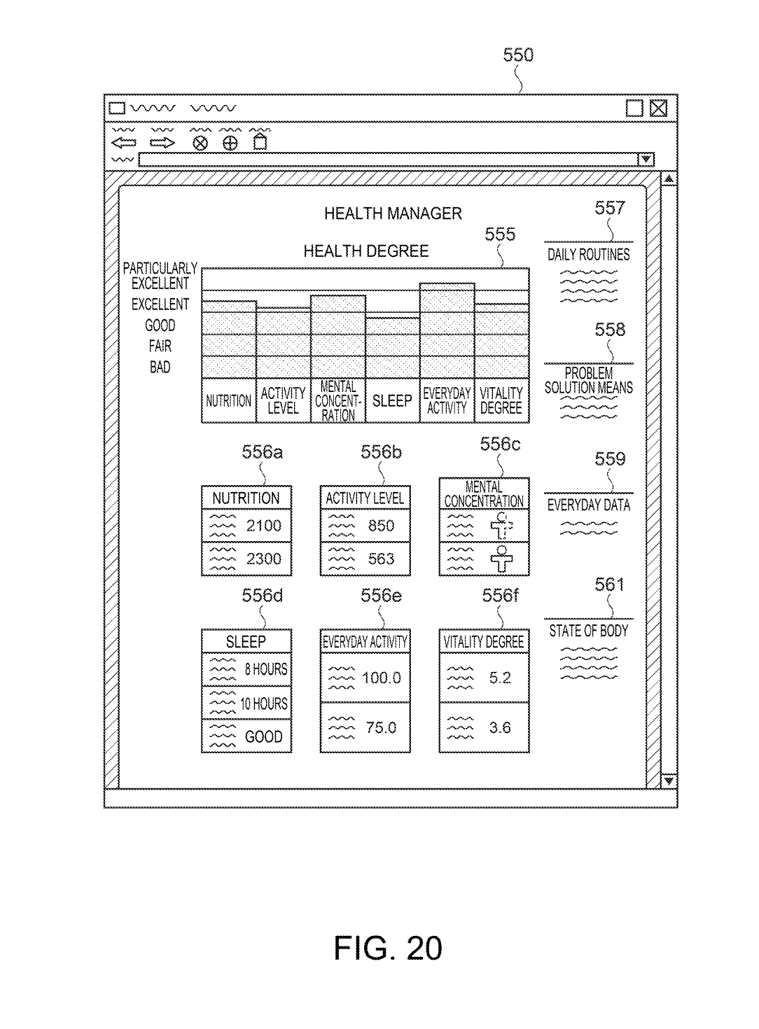

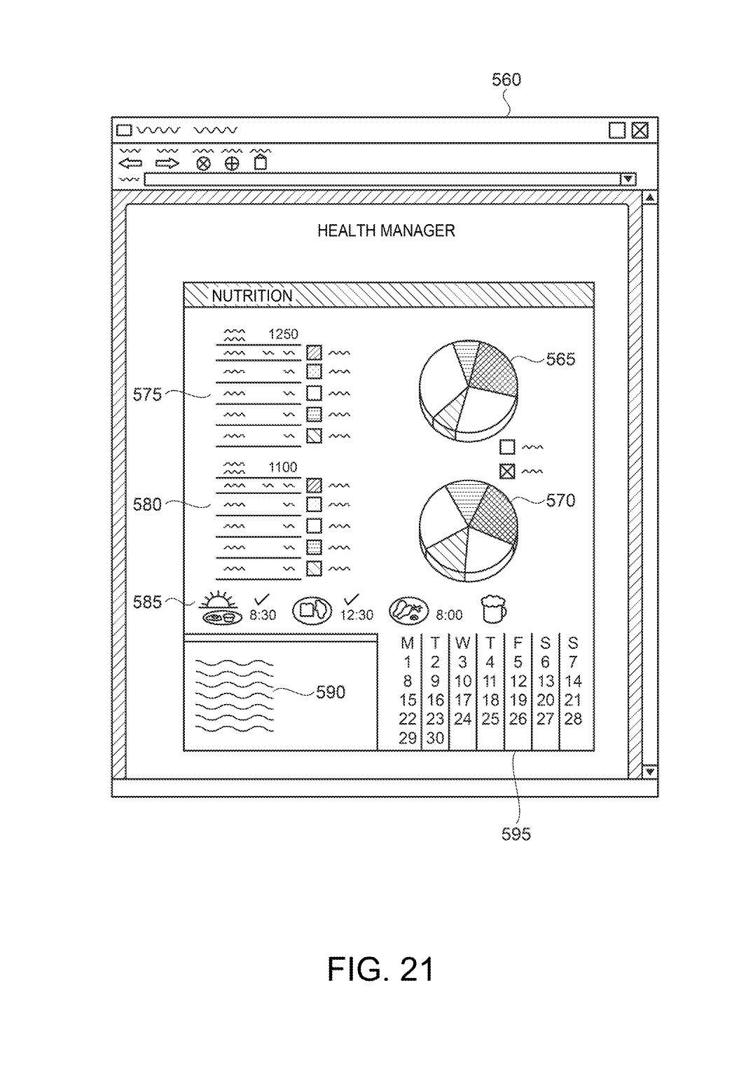

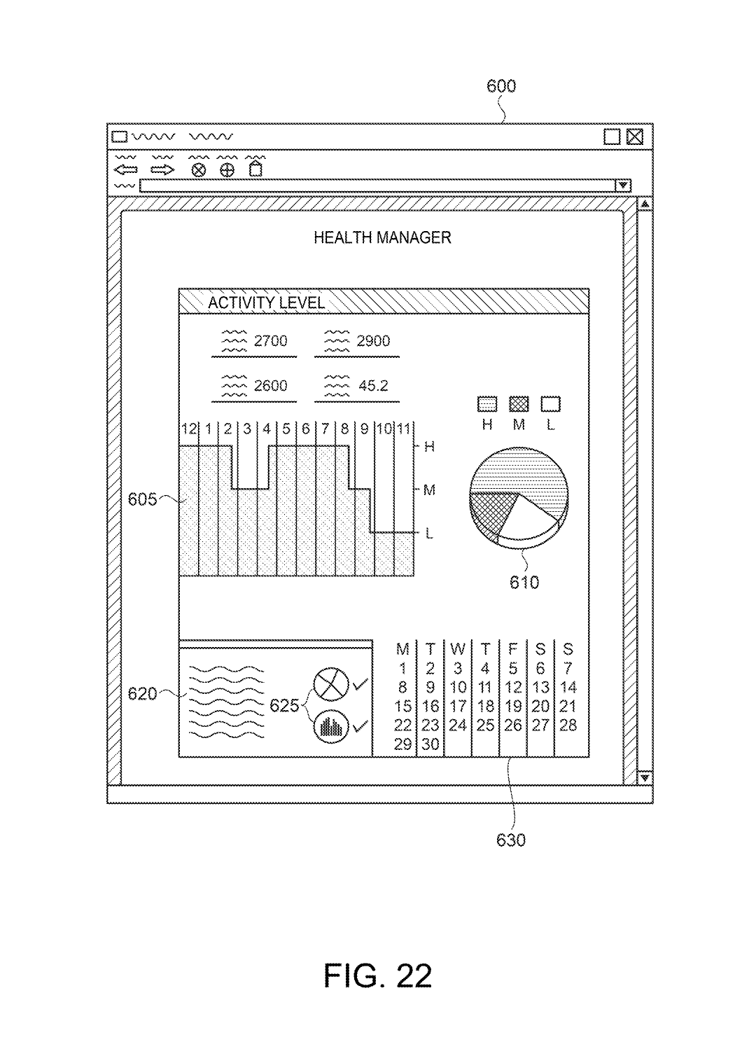

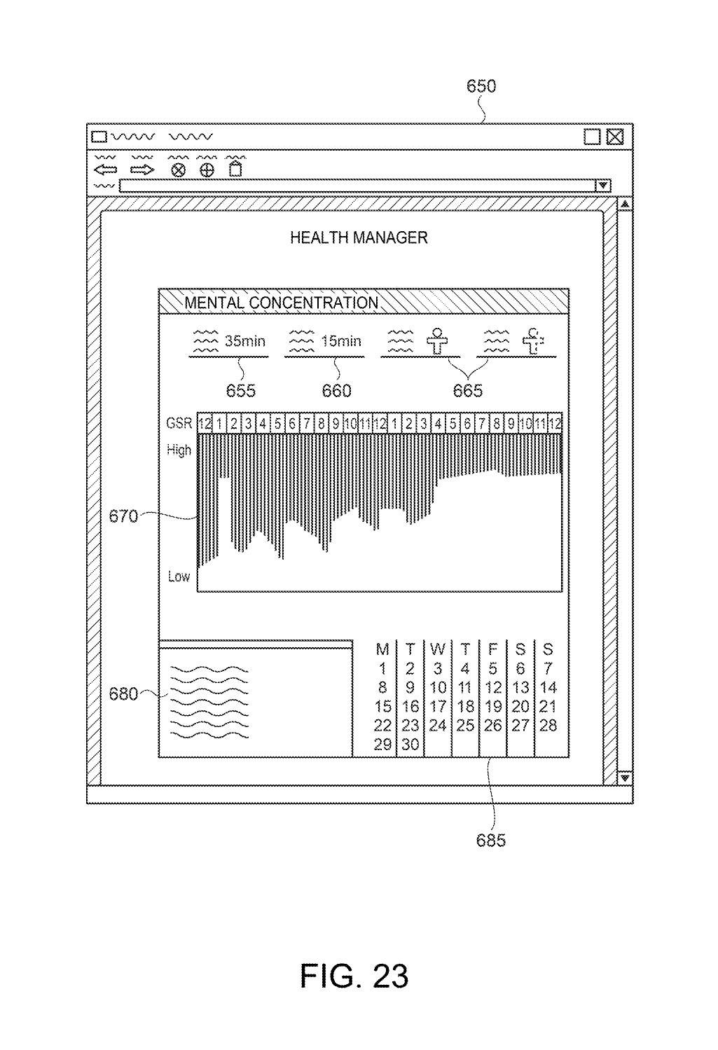

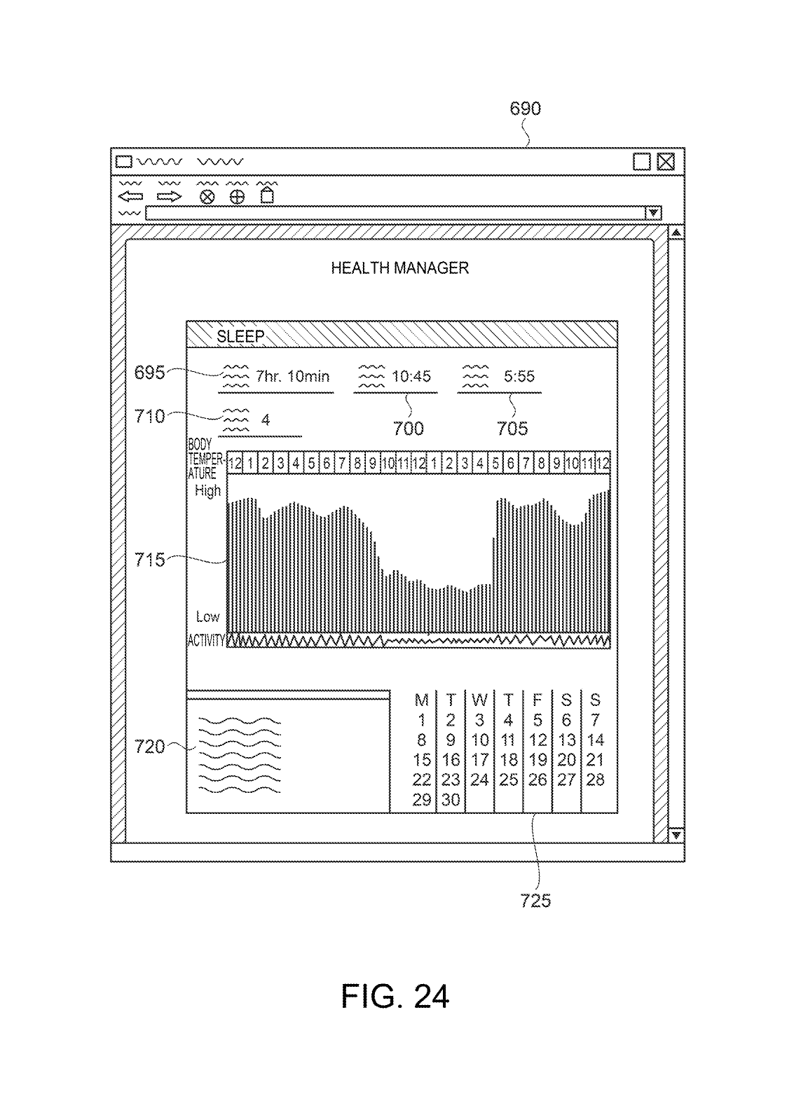

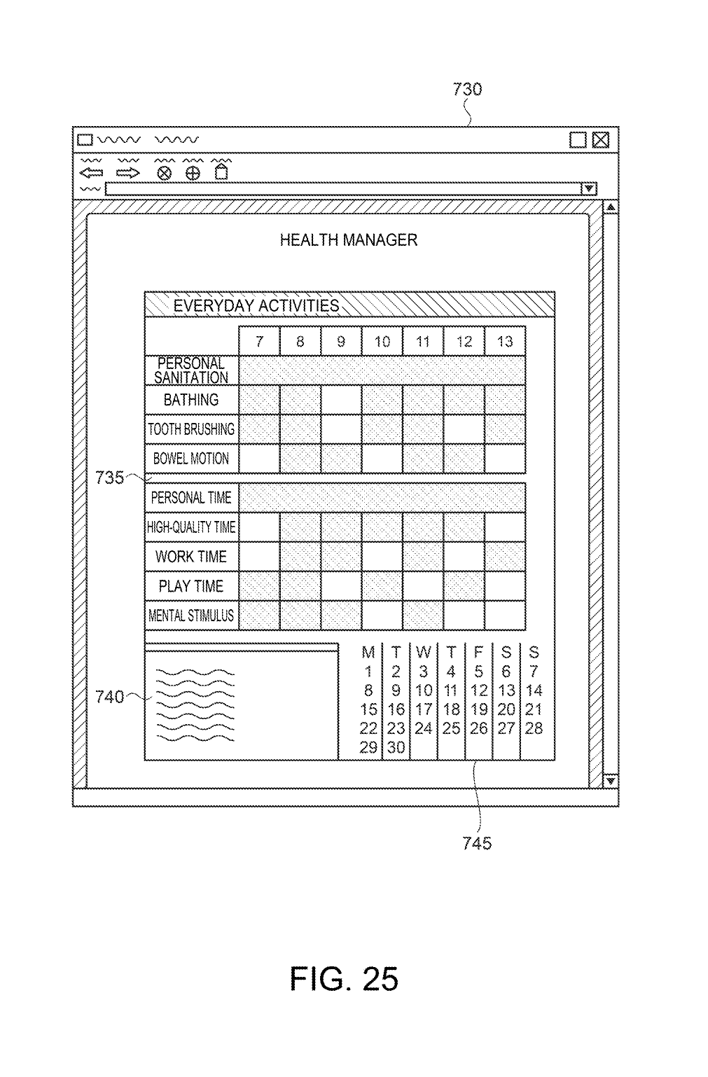

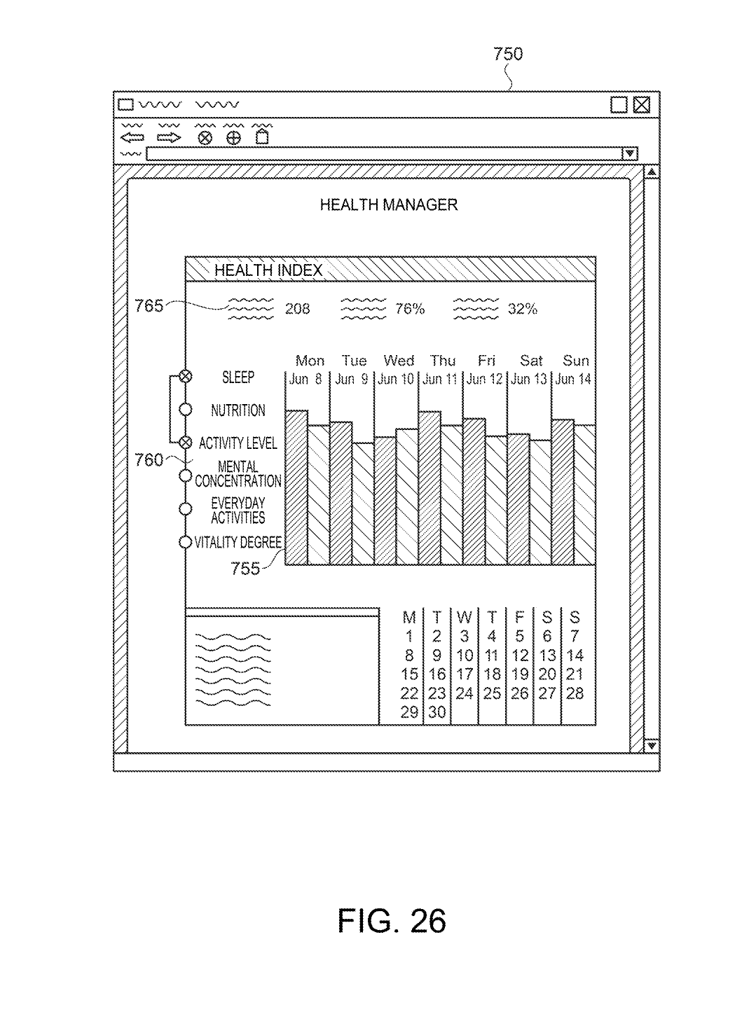

A biological-information measuring device according to an eighth embodiment that can receive provision of characteristic information peculiar to the user and environment information, which need to be treated, in order to maximize the possibility of establishing and maintaining a recommended healthy life style is explained with reference to FIGS. 20 to 26. FIG. 20 is a diagram showing an overview of a Web page serving as a start point of a health manager in the biological-information measuring device according to the eighth embodiment. FIG. 21 is a diagram showing an example of a nutrition Web page. FIG. 22 is a diagram showing an example of an activity level Web page. FIG. 23 is a diagram showing an example of a mental concentration Web page. FIG. 24 is a diagram showing an example of a sleep Web page. FIG. 25 is a diagram showing an example of an everyday activity Web page. FIG. 26 is a diagram showing an example of a health degree Web page.

Although not shown in the figure, the biological-information measuring device according to the eighth embodiment includes, for example, a sensor device connected to a microprocessor. In the biological-information measuring device according to the eighth embodiment, data concerning various life activities finally sent to a monitor unit and stored and personal data or life information input by the user from a Web site maintained by the monitor unit are processed by the microprocessor and provided as biological information. A specific example is explained below.

The user accesses a health manager for the user via a Web page, application software, or other communication media. In FIG. 20, a Web page 550 serving as a start point of the health manager is shown as an example. In the Web page 550 of the health manager shown in FIG. 20, various data are provided to the user. The data provided in this way are one or more of, for example, (1) data indicating various physiological parameters based on values measured by various sensor devices, (2) data derived from the data indicating the various physiological parameters, and (3) data indicating various context parameters generated by the sensor devices and data input by the user.

Analysis state data has a characteristic in using a certain specific utility or algorithm in order to convert one or more of (1) data indicating various physiological parameters acquired by the sensor devices, (2) data derived from the various physiological parameters, and (3) data indicating various context parameters acquired by the sensor devices and data input by the user into a health degree, a robustness degree, a life style index, or the like obtained by calculation. For example, a calorie, amounts of protein, fat, carbohydrate, and a certain specific vitamin, and the like can be calculated on the basis of data input by the user in relation to intake foods. As another example, an index of a stress level for a desired time can be provided to the user by using a skin temperature, a heart rate, a breathing rate, a heat flow, and/or a GSR. As still another example, an index of a sleep pattern for a desired time can be provided to the user by using a skin temperature, a heat flow, a variation between pulsations, a heart rate, a pulse rate, a breathing rate, a center part body temperature, an electric skin reaction, an EMG, an EEG, an EOG, a blood pressure, an oxygen consumption, ambient sound, and a motion of the body detected by a device such as an accelerometer.

On the Web page 550 shown in FIG. 20, a health index 555 serving as a health degree is displayed. The health index 555 is a graphic utility for measuring an achievement of a user and a degree of attainment of healthy daily routines and feeding back the achievement and the degree to member users. In this way, the health index 555 shows, to the member users, health states of the member users and progress states of behaviors concerning health maintenance. The health index 555 includes six categories concerning health and a life style of the user, that is, nutrition, an activity level, mental concentration, sleep, everyday activities, and a vitality degree (a general impression). The category of “nutrition” relates to information concerning what, when, and how much the person (the user) ate and drank. The category of “activity level” relates to an exercise amount indicating how much the person moves around. The category of “mental concentration” relates to the quality (ability) of an activity for changing the person to a relaxed state in a highly concentrated state of the person (the user) and time in which the person concentrates in the activity. The category of “sleep” relates to the quality and the quantity of sleep of the person (the user). The category of “everyday activities” relates to activities the person (the user) has to do every day and health risks that the person encounters. The category of “vitality degree (impression)” relates to a generation perception concerning whether vitality is high in a certain specific day. The categories desirably include level indicators or bar graphs indicating, using scales changing between “bad” and “particularly excellent”, what kinds of achievements the user made concerning the categories.

When the member users end a first investigation explained above, a profile for providing the user with a summary of characteristics of the user and a life environment is created and recommended healthy daily routines and/or targets are presented. The recommended healthy daily routines include any combination of specific advices concerning appropriate nutrition, exercise, mental concentration, and every day activities (life) of the user. An exemplary schedule or the like may be presented as a guide indicating how activities related to the recommended healthy daily routines are adopted in the life of the user. The user periodically takes the investigation and practices the above-mentioned items on the basis of a result of the investigation.

The category of “nutrition” is calculated from both of data input by the user and data sensed by the sensor device. The data input by the user includes hours and drinking and eating times of breakfast, lunch, dinner, and optional snacks, foods to be drunk and eaten, supplements such as vitamins, and water and other liquid (drinking water and liquid foods) to be drunk during time selected in advance. The central monitor unit calculates, on the basis of the data and accumulated data concerning publicly-known characteristics of various foods, well-known nutritional values such as a consumed calorie and contents of protein, fat, carbohydrate, and vitamin.

In the category of “nutrition”, recommended healthy daily routines can be determined on the basis of the bar graph indicating nutrition of the health index 555. The recommended healthy daily routines can be adjusted on the basis of information such as sex, age, and height and weight of the user. Note that the user can set or a substitute of the user can set, on behalf of the user, a calorie to be taken every day, amounts of nutrients such as protein, fiber, fat, and carbohydrate and water, and a target of a certain specific nutrient concerning a ratio to an overall intake amount. Parameters used for the calculation of the bar graphs include the number of times of meals in one day, a consumption of water, and types and amounts of foods eaten every day input by the user.

The nutritional information is presented to the user by a nutrition Web page 560 shown in FIG. 21. The nutrition Web page 560 desirably includes nutrition numerical value charts 565 and 570 respectively indicating actual and target numerical values of nutrition as pie graphs and nutrition intake charts 575 and 580 respectively indicating an actual nutrition intake total amount and a target nutrition intake total amount. The nutrition numerical value charts 565 and 570 desirably indicate items such as carbohydrate, protein, and fat as percentages. The nutrition intake charts 575 and 580 desirably indicate total values and target values of calories separately for components such as fat, carbohydrate, protein, and vitamin. The nutrition Web page 560 also includes a history 585 indicating times in which foods and water were consumed, a hyperlink 590 for enabling the user to directly check news articles related to nutrition, advices for improving daily routines concerning nutrition, and related advertisements somewhere on a network, and a calendar 595 for enabling the user to select an applicable period and the like. Items indicated by the hyperlink 590 can be selected on the basis of information that could have been known concerning an individual through an investigation and an achievement of the individual measured by the health index.

The category of “activity level” of the health index 555 is designed to support a check by the user concerning when and how the user acted (moved) in the day. Both of data input by the user and data sensed by the sensor device are used. The data input by the user includes a detailed item concerning everyday activities of the user indicating that, for example, the user works at a desk from 8:00 am to 5:00 pm and thereafter takes an aerobics lesson from 6:00 pm to 7:00 pm. The related data sensed by the sensor device includes a heart rate, exercise sensed by a device such as an accelerometer, a heat flow, a breathing rate, a consumed calorie amount, a GSR, and a hydration level. These data can be extracted by the sensor device or the central monitor unit. The consumed calorie amount can be calculated by various methods such as a multiplication of a type of exercise input by the user and duration of the exercise input by the user, a multiplication of sensed exercise and time of the exercise and a filter constant, and a multiplication of a sensed heat flow, time, and a filter constant.

In the category of “activity level”, recommended healthy daily routines can be determined on the basis of the bar graph indicating the activity level of the health index 555. The recommended healthy routines are, for example, a minimum target calorie consumed in an activity. Note that the minimum target calorie can be set on the basis of information such as sex, age, height, and weight of the user. Parameters used for the calculation of the bar graph include times consumed for various kinds of exercise and energetic life style activities and input by the user and/or sensed by the sensor device and a calorie burned more than an energy consumption parameter calculated in advance.

Information concerning an activity (a movement) of an individual user is presented to the user by an activity level Web page 600 shown in FIG. 22. The activity level Web page 600 includes an activity degree graph 605 in a form of bar graphs for monitoring activities of the user in three categories, that is, “high”, “medium”, and “low” concerning a predetermined unit time. An activity percentage chart 610 in a form of a pie graph can be presented to indicate percentages in a predetermined period such as one day of consumptions in the respective categories by the user. In the activity level Web page 600, calorie indicators (not shown in the figure) for displaying items such as a burned calorie total amount, an everyday burned calorie target value, a calorie intake total value, and an aerobics exercise time can also be provided. The activity level Web page 600 includes at least one hyperlink 620 for enabling the user to directly check related news articles, advices for improving daily routines concerning an activity level, and related advertisements somewhere on a network.

The activity level Web page 600 can be viewed in various formats. The activity level Web page 600 can enable the user to select a bar graph, a pie graph, or both of the graphs or a chart. The user can select the graph or the chart in an activity level checkbox 625. The activity level calendar 630 is presented to enable the user to select an applicable period and the like. Items shown in the hyperlink 620 can be selected on the basis of information extracted from the individual by an investigation and an achievement measured by the health index.

The category of “mental concentration” of the health index 555 is designed to support the user in monitoring a parameter concerning time in which the user performs an activity for enabling the body to reach a deep relaxed state while concentrating. The category of “mental concentration” is based on both of data input by the user and data sensed by the sensor device. Specifically, the user can input a start time and an end time of a relaxing activity such as yoga or meditation. The quality of these activities determined by the depth of the mental concentration can be measured by monitoring parameters including a skin temperature, a heart rate, a breathing rate, and a heat flow sensed by the sensor device. A percentage change of a GSR obtained by the sensor device or the central monitor unit can also be used.

In the category of “mental concentration”, recommended healthy daily routines can be determined on the basis of a bar graph indicating an activity level of mental concentration of the health index 555. Everyday participation in an activity for deeply relaxing the body while keeping a highly concentrated state is included in the recommended healthy daily routines and displayed. Parameters used for calculation of the bar graph include the length of time consumed for a mental concentration activity, the depth of the mental concentration activity, or a percentage change of a skin temperature, a heart rate, a breathing rate, a heat flow, or a GSR sensed by the sensor device from a baseline indicating quality.

Information concerning time consumed for a behavior for deeply thinking back on the user himself or herself (reflection) and a mental concentration activity for, for example, deeply relaxing the body is presented to the user by a mental concentration Web page 650 shown in FIG. 23. Note that the mental concentration activity is sometimes called session. The mental concentration Web page 650 includes time 655 consumed for the session, a target time 660, a comparison portion 665 indicating a target value and an actual value of the depth of mental concentration, and a histogram 670 indicating an overall stress level derived from, for example, a skin temperature, a heart rate, a breathing rate, a heat flow, and/or a GSR.

In the comparison portion 665, a contour of a human indicating a target mental concentration state is indicated by a solid line. A contour of the human indicating an actual sprit concentration state changes between a blurred state (in FIG. 23, indicated by a broken line) and the solid line according to a level of mental concentration. The mental concentration Web page 650 desirably includes a hyperlink 680 for enabling the user to directly check related news articles on a network, advices and related advertisements for improving daily routines concerning mental concentration, and a calendar 685 for enabling the user to select an applicable period. Items indicated by the hyperlink 680 can be selected on the basis of information that could have been known from an individual through an investigation and an achievement of the individual measured by the health index.

The category of “sleep” of the health index 555 is designed to be capable of supporting the user in monitoring a sleep pattern and the quality of sleep. The category is intended to help the user to learn about the importance of sleep in a healthy life style and a relation of sleep with a daily cycle, which is a normal everyday change of functions of the body. The category of “sleep” is based on both of data input by the user and data sensed by the sensor device. Data input by the user during related time intervals includes bedtime and wakeup time (a sleep time) of the user and a rank of the quality of sleep. Related data obtained from the sensor device includes a skin temperature (a body temperature), a heat flow, a variation between pulsations, a heart rate, a pulse rate, a breathing rate, a center part body temperature, an electric skin reaction, an EMG, an EEG, and EOG, a blood pressure, and an oxygen consumption. Ambient sound and a movement of the body detected by a device such as an accelerometer also have a relation. Thereafter, bedtime and wakeup time, sleep suspension and the quality of sleep, the depth of sleep, and the like can be calculated and derived using the data.

The bar graph indicating sleep of the health index 555 is displayed concerning healthy daily routines including securing of a desirable minimum sleep time of every night, predictable bedtime, and predictable wakeup time. Specific parameters for enabling calculation of the bar graph include bedtime and wakeup time of everyday sensed by the sensor device or input by the user and the quality of sleep graded by the user or derived from other data.

The information concerning sleep is presented to the user by a sleep Web page 690 shown in FIG. 24. The sleep Web page 690 includes a sleep time indicator 695, a user bedtime indicator 700, and a user wakeup time indicator 705 based on data from the sensor device or data input by the user. Note that the quality of sleep input by the user can also be displayed using a sleep quality rank 710. When display exceeding a time interval of one day is performed on the sleep Web page 690, the sleep time indicator 695 can be displayed as a cumulative value and the bedtime indicator 700, the wakeup time indicator 705, and the sleep quality rank 710 can be calculated as average values and displayed. The sleep Web page 690 also includes a sleep graph 715, which is selectable by the user, for calculating and displaying one sleep related parameter over a predetermined time interval. FIG. 24 shows a change in a heat flow (a body temperature) in one day. The heat flow tends to be low during sleep and high when the user is awake. It is possible to obtain a biorhythm of the person from this information.

The sleep graph 715 displays, as a graph, data from an accelerometer built in the sensor device that monitors a movement of the body. The sleep Web page 690 can include a hyperlink 720 for enabling the user to directly check news articles related to sleep, advices for improving daily routines concerning sleep, and related advertisements somewhere on a network and a sleep calendar 725 for selecting a related time interval. Items indicated by the hyperlink 720 can be specially selected on the basis of information that could have been known from an individual through an investigation and an achievement of the individual measured by the health index.

The category of “everyday activities” of the health index 555 is designed to be capable of supporting the user in monitoring a specific activity related to health and safety and a risk and is solely based on data input by the user. Examples of the category of “everyday activities” concerning activities in everyday life include four categories of subordinate concepts. Specifically, the category of “everyday activities” is divided into (1) an item related to personal sanitation for enabling the user to monitor activities for, for example, caring for teeth using a toothbrush or a dental floss and taking a shower, (2) an item related to health maintenance for tracking whether the user drinks a drug or a supplement as prescribed and enabling the user to monitor, for example, consumptions of cigarettes or alcohol, (3) an item related to a personal time for enabling the user to monitor time spent together with a family or friends, leisure, and a mental concentration activity, and (4) an item related to a responsibility for enabling the user to monitor work such as household chores and livelihood activities.

In the category of “everyday activities”, the bar graph indicating “everyday activities” of the health index 555 desirably indicates recommended healthy daily routines explained below. As an example of the daily routine concerning the personal sanitation, the user desirably takes a shower or a bath every day, keeps teeth clean using a brush and a dental floss every day, and maintains a regular bowel motion. As an example of the daily routine concerning the health maintenance, the user desirably drinks a drug, a vitamin tablet, and/or a supplement, stops smoking, drinks alcohol in moderation, and monitors health every day with a health manager. As an example of the daily routine concerning the personal time, the user desirably creates time that user spends together with the family at least for a predetermined time every day and/or spends good time together with friends, reduces time for work, adopts time for leisure or play, and performs intellectual activities. As an example of the daily routine concerning the responsibility, the user desirably performs household chores, is not late for work, and keeps a promise. The bar graph is determined according to information input by the user and/or calculated on the basis of a degree of the user completing listed activities every day.

Information concerning these activities is presented to the user by an everyday activity Web page 730 shown in FIG. 25. An activity chart 735 in the everyday activity Web page 730 indicates whether the user executed the activities required by the daily routines. The activity chart 735 can be selected concerning one or more of subordinate concepts. In the activity chart 735, colored or shaded boxes indicate that the user executed the required activities and uncolored or unshaded boxes indicate that the user did not execute the activities. The activity chart 735 can be created and viewed in a selectable time interval. FIG. 25 shows, as an example, the categories of the personal sanitation and the personal time in a specific week. Further, the everyday activity Web page 730 can include a hyperlink 740 for enabling the user to directly check related news articles, advices for improving daily routines concerning activities of everyday life, and related advertisements somewhere on a network and a calendar 745 of everyday activities for selecting a related time interval. Items indicated by the hyperlink 740 can be selected on the basis of information that could have been known from an individual in an investigation and an achievement determined by the health index.

The category of “vitality degree” of the health index 555 is designed to enable the user to monitor recognition concerning whether the user was fine in a specific day and is based on essentially subjective grade information directly input by the user. The user performs ranking desirably using scales 1 to 5 concerning the following nine areas, i.e., (1) mental sharpness, (2) mental and psychological happiness degrees, (3) an energy level, (4) an ability to cope with stress of life, (5) a degree of putting importance on a reputation, (6) a physical happiness degree, (7) self-constraint, (8) a motivation, and (9) a comfort through a relation with others. These degrees (ranks) are averaged and used for calculation of the bar graph of the health index 555.

FIG. 26 shows a vitality degree Web page 750. The vitality degree Web page 750 enables the user to check vitality degrees over a time interval selectable by the user including continuous or discontinuous any days. Note that, in an example shown in FIG. 26, the vitality degrees are displayed as health indexes. On the vitality degree Web page 750, by using a selection box 760 of the vitality degrees, the user can perform selection for checking bar graph 755 of the vitality degree concerning one category or arrange bar graphs 755 of the vitality degrees side by side and compare the bar graphs 755 concerning two categories or two or more categories. For example, the user sometimes desires to set only a bar graph of sleep in an active state in order to check whether a general rank of sleep is improved compared with the preceding month or sometimes simultaneously displays sleep and activity levels to thereby compare and evaluate a grade of sleep and a grade of an activity level corresponding to the grade of sleep and check whether some correlation is present among the days. The user sometimes displays a grade of nutrition and a grade of a vitality degree concerning a predetermined time interval and checks whether some correlation is present between an everyday meal habit and a meal habit and a vitality degree during the interval. FIG. 26 shows, as an example for explanation, comparison of sleep and activity levels in a week of June 8 to June 14 by bar graphs. The vitality degree Web page 750 also includes a track calculator 765 that displays access information such as a total number of days in which the user logged in and used the health manager, and a ratio of days in which the user used the health manager after becoming a member, and a ratio of time in which the user used the sensor device in order collect data, and statistics.

An example of the Web page 550 serving as a start point of the health manager shown in FIG. 20 includes summaries 556a to 556f of a plurality of categories selectable by the user respectively corresponding to the categories of the health index 555 serving as health degrees. The summaries 556a to 556f of the categories present subsets of data selected and filtered in advance concerning the corresponding categories. The summary 556a of the nutrition category indicates a target value and an actual value of every day of a calorie intake amount. The summary 556b of the activity level category indicates a target value and an actual value of every day of a burned calorie amount. The summary 556c of the mental concentration category indicates a target value and an actual value of the depth of mental concentration. The summary 556d of the sleep category indicates a target sleep time, an actual sleep time, and a grade of the quality of sleep. The summary 556e of the everyday activity category indicates a target point and an actual point based on a ratio of completed activities to recommended healthy daily routines (everyday activities). The summary 556f of the vitality degree category indicates a target grade and an actual grade of a vitality degree in the day.

The Web page 550 can also include a hyperlink (not shown in the figure) to news articles, a comment (not shown in the figure) to the user based on a tendency such as undernourishment checked by a first investigation, and a sign (not shown in the figure). The Web page 550 can also include an everyday routine portion 557 for providing the user with information every day. As a comment of the everyday routine portion 557, for example, a water intake needed every day and an advice of specific means for enabling the water intake can be displayed. The Web page 550 can include a problem solution section 558 for actively evaluating the performance of the user in the categories of the health index 555 and presenting an advice for improvement. For example, when a system indicates that a sleep level of the user is “low” and the user has insomnia, the problem solution section 558 can advise a method for improving sleep. The problem solution section 558 can include a question of the user concerning improvement of achievement. The Web page 550 can include an everyday data section 559 for starting an input dialog box. With the input dialog box, the user can easily perform an input of various data required by the health manager. As it is known in the technical field, it is possible to select whether the input of the data is an input of a list presented in advance or an input in a normal free text format. The Web page 550 can include a body state section 561 for giving information concerning a life symptom such as height, weight, body measurement values, a BMI, and a heart rate, a blood pressure or any physiological parameters of the user.

### Modification of the Light Receiver

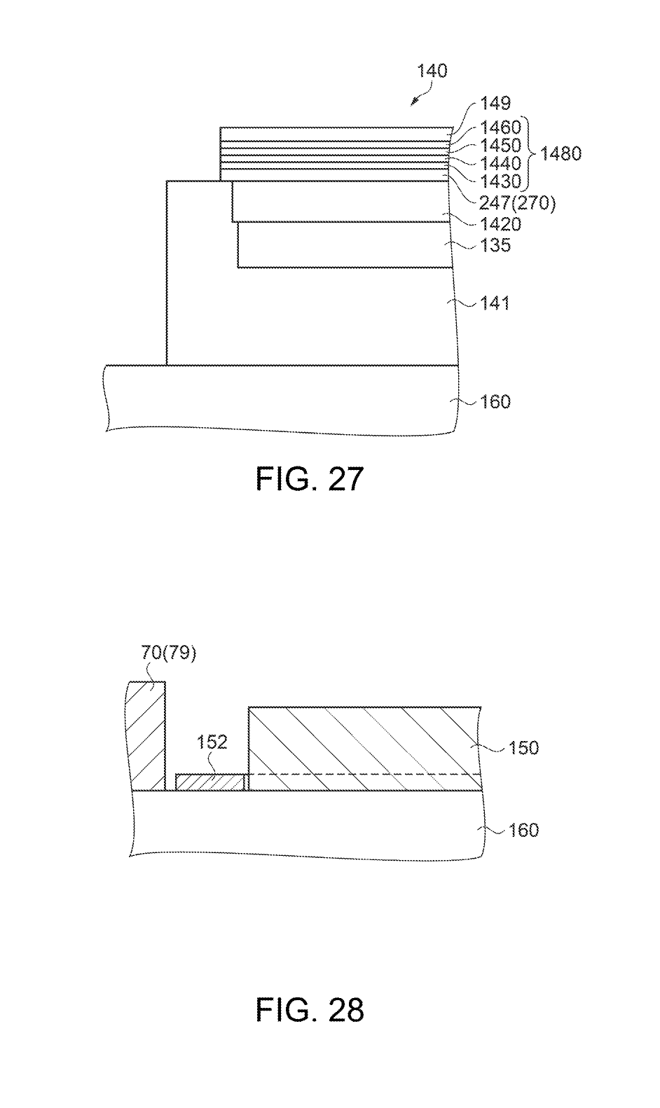

A modification of the light receiver 140 according to the first embodiment is explained with reference to FIG. 27. FIG. 27 is a partial sectional view showing the modification of the light receiver. As shown in FIG. 27, the light receiver 140 mounted on the substrate 160 (the sensor substrate) can be realized by a photodiode element 135 or the like of PN junction formed on the semiconductor substrate 141. In this case, an angle limiting filter (the first convex sections 247) for narrowing a light reception angle and a wavelength limiting filter (an optical filter film) 1480 for limiting a wavelength of light made incident on the light receiving element may be provided on the photodiode element 135 or the protection layer 1420 provided on the photodiode element 135. In this modification, the angle limiting filter (the first convex sections 247) is provided on the protection layer 1420 provided on the photodiode element 135. The wavelength limiting filter (the optical filter film) 1480 is provided on the upper side of the angle limiting filter (the first convex sections 247). Note that, in the wavelength limiting filter (the optical filter film) 1480, for example, a first oxide film 1430, a first nitride film 1440, a second oxide film 1450, and a second nitride film 1460 are formed in this order from the angle limiting filter (the first convex section 247) side. A resin film 149 having light transmissivity is provided on the wavelength limiting filter (the optical filter film) 1480.

By adopting such a configuration, with the resin film 149 having light transmissivity provided on the wavelength limiting filter (the optical filter film) 1480, it is possible to improve a waterproof property and an antifouling property of the wavelength limiting filter (the optical filter film) 1480.

Note that the modification of the light receiver is applicable in all of the embodiments or the configuration examples explained above.

### Modification of the Light Emitter

A modification of the first light emitter 150 according to the first embodiment is explained with reference to FIG. 28. FIG. 28 is a partial sectional view showing the modification of the light emitter. As shown in FIG. 28, around the first light emitter 150 mounted on the substrate 160 (the sensor substrate), the first wall section 70 functioning as the frame and a reflection function layer 152 that reflects light emitted from the first light emitter 150 in the peripheral direction are provided. Note that the reflection function layer 152 may be provided to surround the entire periphery of the first light emitter 150 or may be provided at least in a part of the periphery of the first light emitter 150 in plan view of the substrate 160 viewed from the upper surface side.

By adopting such a configuration, it is possible to reflect, with the reflection function layer 152, the light emitted in the peripheral direction of the first light emitter 150 and change the light to light traveling to a measurement target object. Consequently, it is possible to increase the intensity (light emission intensity) of the light traveling to the measurement target object. It is possible to improve and stabilize measurement accuracy of biological information.

Note that the modification of the light emitter is applicable in all of the embodiments or the configuration examples explained above.

Not that the embodiments are explained above in detail. Those skilled in the art could easily understand that many modifications are possible without substantially departing from the new matters and the effects of the invention. Therefore, all such modifications are deemed to be included in the scope of the invention. For example, terms described together with broader or synonymous different terms at least once in the specification or the drawings can be replaced with the different terms in any place in the specification or the drawings. The configurations and operations of the biological-information measuring module, the light detecting unit, the biological-information measuring device, and the like are not limited to the configurations explained in the embodiments. Various modified implementations of the configurations are possible.

What is claimed is:

1. A pulse-wave measuring module comprising:
a light emitter configured to emit light to a test object; and
a light receiver configured to receive reflected light from the test object, wherein
the light receiver includes:
a light detector configured to detect the reflected light; and
an optical filter disposed on the light detector and configured by a plurality of layers, and
a layer most distant from the light detector among the plurality of layers of the optical filter is made of silicon oxide.

2. The pulse-wave measuring module according to claim 1, wherein the optical filter is configured by the layer made of the silicon oxide and a layer made of silicon nitride.

3. The pulse-wave measuring module according to claim 2, wherein
a layer nearest from the light detector of the optical filter is the layer made of the silicon oxide, and
the layer made of the silicon oxide and the layer made of the silicon nitride are alternately stacked.

4. The pulse-wave measuring module according to claim 2, wherein thickness of the layer made of the silicon nitride is smaller than thickness of the layer made of the silicon oxide.

5. The pulse-wave measuring module according to claim 2, wherein thickness of the layer made of the silicon nitride is larger than thickness of the layer made of the silicon oxide.

6. The pulse-wave measuring module according to claim 1, wherein thickness of the optical filter is 0.7 μm or more and 1.0 μm or less.

7. The pulse-wave measuring module according to claim 1, wherein thickness of the optical filter is 0.1 μm or more and 0.4 μm or less.

8. The pulse-wave measuring module according to claim 1, wherein the light receiver is sealed by transparent resin.

9. The pulse-wave measuring module according to claim 8, wherein the transparent resin comes into contact with the test object.

10. The pulse-wave measuring module according to claim 1, further comprising a wall section disposed between the light emitter and the light receiver, wherein
the wall section further projects to the test object side than the light emitter and the light receiver.

11. The pulse-wave measuring module according to claim 10, further comprising a frame section including the wall section and surrounding the light receiver, wherein
an upper end face of the frame section is higher than an upper surface of the light receiver.

12. An electronic device mounted with the pulse-wave measuring module according to claim 1.

13. A biological-information measuring module comprising:
a first light emitter;
a light receiver; and
a filter section including a plurality of first convex sections disposed on a light receiving surface of the light receiver, arranged side by side along a first direction from a center of the first light emitter to a center of the light receiver in plan view from a direction perpendicular to the light receiving surface, and projecting from the light receiving surface.

14. The biological-information measuring module according to claim 13, wherein the first convex sections extend along the first direction.

15. The biological-information measuring module according to claim 13, wherein height from the light receiving surface of an end portion convex section located at an end portion of the filter section among the plurality of first convex sections is larger than height from the light receiving surface of the other first convex sections.

16. The biological-information measuring module according to claim 13, further comprising a first wall section disposed between the light receiver and the first light emitter, wherein
height of the first wall section is larger than height of the first convex sections.

17. The biological-information measuring module according to according to claim 13, wherein the first convex sections are configured by a stacked body of functional layers.

18. The biological-information measuring module according to claim 13, further comprising a second light emitter, wherein
the filter section includes a plurality of second convex sections disposed on the light receiving surface of the light receiver, arranged side by side along a second direction from a center of the second light emitter to the center of the light receiver in plan view from a direction perpendicular to the light receiving surface, and projecting from the light receiving surface.

19. The biological-information measuring module according to claim 18, wherein the second convex sections extend along the second direction.

20. The biological-information measuring module according to claim 18, further comprising a second wall section disposed between the light receiver and the second light emitter, wherein
height of the second wall section is larger than height of the second convex sections.
